(19) United States
(12) Patent Application Publication
(10) Pub. No.: US 2011/0111396 A1
(43) Pub. Date: May 12, 2011

(54) BIOMARKERS FOR MONITORING THE TREATMENT BY QUINAZOLINONE COMPOUNDS

(76) Inventors: Peter H. Schafer, Somerset, NJ (US); Justin . Bartlett, Warren, NJ (US); Ling-Hua Zhang, Parsippany, NJ (US)

(21) Appl. No.: 12/746,503

(22) PCT Filed: Dec. 5, 2008
(86) PCT No.: PCT/US08/13444

§ 371 Date: Jan. 14, 2011

Related U.S. Application Data
(60) Provisional application No. 61/005,804, filed on Dec. 7, 2007.

(51) Int. Cl. C12Q 1/68

(52) U.S. Cl. 435/6

(57) ABSTRACT

Provided herein are the biomarkers for monitoring the treatment by quinazolinone compounds. For example, the use of SPARC, p21, and cyclin D1 mRNA levels as biomarkers to predict whether a quinazolinone compound is likely to be successful in treating certain types of cancer, such as NHL is provided. Further, the expression of these genes can be used to monitor progress of treatment effectiveness and patient compliance in cancer patients that are receiving treatment with quinazolinone compounds.

This application claims priority to U.S. Provisional Application No. 61/005,804, filed Dec. 7, 2007, the entirety of which is incorporated herein by reference.

### 1. FIELD

This invention relates to the monitoring of expression of a specific set of genes or proteins before and during therapy with a quinazolinone compound to treat cancer, e.g., non-Hodgkin's lymphoma patients.

### 2. BACKGROUND

Various compounds have been used to treat cancer. An example is a group of quinazolinone compounds previously described in, e.g., U.S. Patent Publication No. US 2008/0161328, published Jul. 3, 2008, and U.S. application Ser. No. 12/238,354, filed Sep. 25, 2008, both of which are incorporated herein by reference in their entireties.

### 3. SUMMARY

Provided herein is the use of specific mRNAs and proteins as biomarkers to ascertain the effectiveness and progress of the treatment by quinazolinone compounds. For example, the mRNA or protein levels of SPARC, p21, and cyclin D1 can be used to determine whether a quinazolinone compound is likely to be successful in treating certain types of cancer, such as NHL. Further, the expression of these genes or proteins can be used to monitor progress of treatment effectiveness in NHL patients that are receiving treatment with quinazolinone compounds.

In some embodiments, a method of predicting tumor response to treatment in a Non-Hodgkin's Lymphoma (NHL) patient is provided. The method comprises obtaining tumor cells from the patient, culturing the cells in the presence or absence of a quinazolinone compound, measuring SPARC expression in the tumor cells, and comparing the levels of SPARC expression level in tumor cells cultured in the presence of the quinazolinone compound to those in tumor cells cultured in the absence of the compound, wherein an increased level of SPARC expression in the presence of the quinazolinone compound indicates the likelihood of an effective patient tumor response to the compound.

In another embodiment, a method of monitoring tumor response to treatment in a Non-Hodgkin's Lymphoma (NHL) patient is provided. The method comprises obtaining a biological sample from the patient, measuring SPARC expression in the biological sample, administering a quinazolinone compound to the patient, thereafter obtaining a second biological sample from the patient, measuring SPARC expression in the second biological sample, and comparing the levels of SPARC expression, where an increased level of SPARC expression after treatment indicates the likelihood of an effective tumor response.

In yet another embodiment, a method for monitoring patient compliance with a drug treatment protocol is provided. The method comprises obtaining a biological sample from the patient, measuring the expression level of at least one of p21, cyclin D1, or SPARC in the sample, and determining if the expression level is increased or decreased in the patient sample compared to the expression level in a control untreated sample, wherein an increased or decreased expression indicates patient compliance with the drug treatment protocol. In one embodiment, the expression of SPARC is monitored. In another embodiment, an increase in the expression of SPARC indicates the compliance.

The expression monitored can be, for example, mRNA expression or protein expression. The expression in the treated sample can increase, for example, by about 1.5×, 2.0×, 3×, 5×, or more.

In another embodiment, a method of predicting the sensitivity to treatment with a quinazolinone compound in an NHL, specifically, a Mantle Cell Lymphoma (MCL), patient is provided. The method comprises obtaining a biological sample from the patient, optionally isolating or purifying mRNA from the biological sample, amplifying the mRNA transcripts by, e.g., RT-PCR, and comparing the cycle number at which the fluorescence passes the set threshold level (“CT”) of Cyclin D1 and P21 mRNA expression, where a greater difference between P21 CT and Cyclin D1 CT (dCT) indicates a higher likelihood that the cancer will be sensitive to treatment with the quinazolinone compound. The difference between P21 CT and Cyclin D1 CT can be, for example, higher than 0. The difference between P21 CT and Cyclin D1 CT can be, for example, higher than about 1, 2, 3, 4, 5, 6, 7, 8, 9, or 10.

In another embodiment, a method of predicting the sensitivity to treatment with an immunomodulatory compound in an NHL, specifically, a Mantle Cell Lymphoma (MCL), patient is provided. The method comprises obtaining a biological sample from the patient, optionally isolating or purifying mRNA from the biological sample, amplifying the mRNA transcripts by, e.g., RT-PCR, where a higher baseline level of Cyclin D1 (as assessed by, e.g., determining the cycle number at which the fluorescence passes the set threshold level (“CT”) of Cyclin D1 mRNA expression) indicates a higher likelihood that the cancer will be sensitive to treatment with an immunomodulatory compound.

In yet another embodiment, a kit useful for predicting the likelihood of an effective treatment of NHL with a quinazolinone compound is provided. The kit comprises a solid support, nucleic acids contacting the support, where the nucleic acids are complementary to at least 20, 50, 100, 200, 350, or more bases of at least one of 1) cyclin D1 mRNA and 2) p21 mRNA, and a means for detecting the expression of the mRNA in a biological sample.

In an additional embodiment, a kit useful for predicting the likelihood of an effective NHL treatment or for monitoring the effectiveness of a treatment with a quinazolinone compound is provided. The kit comprises a solid support, at least one nucleic acid contacting the support, where the nucleic acid is complementary to at least 20, 50, 100, 200, 350, 500, or more bases of SPARC mRNA, and a means for detecting the expression of the mRNA in a biological sample.

In an additional embodiment, a kit useful for predicting the likelihood of an effective treatment of NHL or for monitoring treatment with a quinazolinone compound is provided. The kit comprises a solid support, and a means for detecting the protein expression of at least one of SPARC, cyclin D1, and p21 in a biological sample.

Such a kit can employ, for example a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample. The biological sample can be, for example, a lymph node biopsy, a bone marrow biopsy, or a sample of peripheral blood tumor cells.

### 4. BRIEF DESCRIPTION OF FIGURES

FIG. 1 illustrates the inhibition of cell proliferation by a quinazolinone compound 3-(2,5-dimethyl-4-oxo-4H-quinazolin-3-yl)-piperidine-2,6-dione at 0.01 μM, 0.1 μM, 1 μM, 10 μM, and 100 μM concentrations, as observed in Namalwa, Rec-1, Jeko-1, Granta-519, JVM-2, and DB cell lines.

FIG. 2 illustrates the changes in the level of SPARC mRNA in Namalwa, Rec-1, Jeko-1, Granta-519, JVM-2, and DB cell lines upon treatment by 10 μM of 3-(2,5-dimethyl-4-oxo-4H-quinazolin-3-yl)-piperidine-2,6-dione.

### 5. DETAILED DESCRIPTION

Subject matter provided herein is based, in part, on the discovery that the presence and level of certain mRNAs or proteins in cell samples can be utilized as biomarkers to indicate the effectiveness or progress of a disease treatment. In particular, these mRNA or protein biomarkers can be used to predict, assess and track the effectiveness of patient treatment with various quinazolinone compounds.

Without being limited to a particular theory, quinazolinone compounds can mediate growth inhibition, apoptosis and inhibition of angiogenic factors in certain types of cancer such as NHL. Upon examining the expression of several cancer-related genes or proteins in several cell types before and after the treatment with a quinazolinone compound, it was discovered that the expression levels of certain cancer-related genes or proteins can be used as biomarkers for predicting and monitoring cancer treatments.

5.1 Definitions

So that the invention is more fully understood, the following terms are more clearly defined:

As used herein, and unless otherwise specified, the terms “treat,” “treating” and “treatment” refer to an action that occurs while a patient is suffering from the specified cancer, which reduces the severity of the cancer, or retards or slows the progression of the cancer.

The term “sensitivity” and “sensitive” when made in reference to treatment with a quinazolinone compound is a relative term which refers to the degree of effectiveness of the quinazolinone compound in lessening or decreasing the progress of a tumor or the disease being treated. For example, the term “increased sensitivity” when used in reference to treatment of a cell or tumor in connection with a quinazolinone compound refers to an increase of, at least a 5%, or more, in the effectiveness of the tumor treatment.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

As used herein, an “effective patient tumor response” refers to any increase in the therapeutic benefit to the patient. An “effective patient tumor response” can be, for example, a 5%, 10%, 25%, 50%, or 100% decrease in the rate of progress of the tumor. An “effective patient tumor response” can be, for example, a 5%, 10%, 25%, 50%, or 100% decrease in the physical symptoms of a cancer. An “effective patient tumor response” can also be, for example, a 5%, 10%, 25%, 50%, 100%, 200%, or more increase in the general health of the patient, as measured by any suitable means, such as gene expression, cell counts, assay results, etc.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the effectiveness of a quinazolinone compound” refers to tracking the effectiveness in treating a cancer in a patient or in a tumor cell culture. Similarly, the “monitoring,” when used in connection with patient compliance, either individually, or in a clinical trial, refers to the tracking or confirming that the patient is actually taking the i compound being tested as prescribed. The monitoring can be performed, for example, by following the expression of mRNA or protein biomarkers such as SPARC, cyclin D1, p21, and mRNAs thereof.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to, lymphoma and leukemia, and solid tumors.

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. Exemplary polypeptides disclosed herein include, but are not limited to, SPARC, cyclin D1, p21, and the like. The polypeptide can be purified from a biological sample.

The term “antibody” is used herein in the broadest sense and covers fully assembled antibodies, antibody fragments which retain the ability to specifically bind to the antigen {e.g., Fab, F(ab′)2, Fv, and other fragments), single chain antibodies, diabodjes, antibody chimeras, hybrid antibodies, bispecific antibodies, humanized antibodies, and the like. The term “antibody” covers both polyclonal and monoclonal antibodies.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide or a portion thereof.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition.

An mRNA from a patient sample can be “upregulated” when treated with quinazolinone compound, as compared to a non-treated control. This upregulation can be, for example, an increase of about 5%, 10%, 20%, 30%, 40%, 50%, 60%, 70%, 90%, 100%, 200%, 300%, 500%, 1,000%, 5,000% or more of the comparative control mRNA level.

Alternatively, an mRNA can be “downregulated”, or expressed at a lower level, in response to administration of certain quinazolinone compounds or other agents. A downregulated mRNA can be, for example, present at a level of about 99%, 95%, 90%, 80%, 70%, 60%, 50%, 40%, 30%, 20%, 10%, 1% or less of the comparative control mRNA level.

Similarly, the level of a polypeptide or protein biomarker from a patient sample can be increased when treated with a quinazolinone compound, as compared to a non-treated control. This increase can be about 5%, 10%, 20%, 30%, 40%, 50%, 60%, 70%, 90%, 100%, 200%, 300%, 500%, 1,000%, 5,000% or more of the comparative control protein level.

Alternatively, the level of a protein biomarker can be decreased in response to administration of certain quinazolinone compounds or other agents. This decrease can be, for example, present at a level of about 99%, 95%, 90%, 80%, 70%, 60%, 50%, 40%, 30%, 20%, 10%, 1% or less of the comparative control protein level.

The terms “determining,” “measuring,” “evaluating,” “assessing,” and “assaying,” as used herein, generally refer to any form of measurement, and include determining if an element is present or not. These terms include both quantitative and/or qualitative determinations. Assessing may be relative or absolute. “Assessing the presence of can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C, although small regions (e.g. less than about 3 bases) of mismatch, insertion, or deleted sequence may be present.

“Sequence identity” or “identity” in the context of two nucleic acid sequences refers to the residues in the two sequences which are the same when aligned for maximum correspondence over a specified comparison window, and can take into consideration additions, deletions and substitutions.

The term “substantial identity” or “homologous” in their various grammatical forms in the context of polynucleotides generally means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence. Another indication that nucleotide sequences are substantially identical is if two molecules hybridize to each other under stringent conditions.

As used herein, the term “bound” can be used herein to indicate direct or indirect attachment. In the context of chemical structures, “bound” (or “bonded”) may refer to the existence of a chemical bond directly joining two moieties or indirectly joining two moieties (e.g. via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect.

The terms “isolated” and “purified” refer to isolation of a substance (such as mRNA or protein) such that the substance comprises a substantial portion of the sample in which it resides, i.e., greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, and the like.

The term “analyte” as used herein, refers to a known or unknown component of a sample.

The term “capture agent,” as used herein, refers to an agent that binds an mRNA or protein through an interaction that is sufficient to permit the agent to bind and concentrate the mRNA or protein from a homogeneous mixture.

The term “probe” as used herein, refers to a capture agent that is directed to a specific target mRNA biomarker sequence. Accordingly, each probe of a probe set has a respective target mRNA biomarker. A probe/target mRNA duplex is a structure formed by hybridizing a probe to its target mRNA biomarker.

The term “nucleic acid” or “oligonucleotide probe” refers to a nucleic acid capable of binding to a target nucleic acid of complementary sequence, such as the mRNA biomarkers provided herein, through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term stringent assay conditions generally refers to the combination of hybridization and wash conditions.

A “label” or a “detectable moiety” in reference to a nucleic acid, refers to a composition that, when linked with a nucleic acid, renders the nucleic acid detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

The terms “Polymerase chain reaction,” or “PCR,” as used herein generally refers to a procedure wherein small amounts of a nucleic acid, RNA and/or DNA, are amplified as described, for example, in U.S. Pat. No. 4,683,195 to Mullis. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, NY, 1989).

The term “cycle number” or “CT” when used herein in reference to PCR methods, refers to the PCR cycle number at which the fluorescence level passes a given set threshold level. The CT measurement can be used, for example, to approximate levels of mRNA in an original sample. The CT measurement is often used in terms of “dCT” or the “difference in the CT” score, when the CT of one nucleic acid is subtracted from the CT of another nucleic acid.

As used herein, and unless otherwise indicated, the term “optically pure” means a composition that comprises one optical isomer of a compound and is substantially free of other isomers of that compound. For example, an optically pure composition of a compound having one chiral center will be substantially free of the opposite enantiomer of the compound. An optically pure composition of a compound having two chiral centers will be substantially free of other diastereomers of the compound. A typical optically pure compound comprises greater than about 80% by weight of one enantiomer of the compound and less than about 20% by weight of other enantiomers of the compound, more preferably greater than about 90% by weight of one enantiomer of the compound and less than about 10% by weight of the other enantiomers of the compound, even more preferably greater than about 95% by weight of one enantiomer of the compound and less than about 5% by weight of the other enantiomers of the compound, more preferably greater than about 97% by weight of one enantiomer of the compound and less than about 3% by weight of the other enantiomers of the compound, and most preferably greater than about 99% by weight of one enantiomer of the compound and less than about 1% by weight of the other enantiomers of the compound.

The practice of the present invention will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al. (1989) Molecular Cloning; A Laboratory Manual (2d ed.); D. N Glover, ed. (1985) DNA Cloning, Volumes I and II; M. J. Gait, ed. (1984) Oligonucleotide Synthesis; B. D. Hames & S J. Higgins, eds. (1984) Nucleic Acid Hybridization; B. D. Hames & S. J. Higgins, eds. (1984) Transcription and Translation; R. I. Freshney, ed. (1986) Animal Cell Culture; Immobilized Cells and Enzymes (IRL Press, 1986); Immunochemical Methods in Cell and Molecular Biology (Academic Press, London); Scopes (1987) Protein Purification: Principles and Practice (2d ed.; Springer Verlag, N.Y.); and D. M. Weir and C. C. Blackwell, eds. (1986) Handbook of Experimental Immunology, Volumes I-IV.

5.2 Non-Hodgkin's Lymphoma

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment (Freedman et al., “Non-Hodgkin's Lymphomas” Chapter 134, Cancer Medicine, (an approved publication of the American Cancer Society, B. C. Decker Inc., Hamilton, Ontario, 2003).

Examples of one type of lymphoma, non-Hodgkin's lymphoma, include but are not limited to Adult T-Cell Lymphoma/Leukemia (ATLL), Anaplastic Large Cell Lymphoma (ALCL), Angiocentric Nasal T-Cell Lymphoma, Angiocentric Pulmonary B-Cell Lymphoma, Angioimmunoblastic Lymphoma, Burkitt's Lympoma (See Small Non-Cleaved Cell Lymphoma), Centrocytic Lymphoma (see Mantle Cell Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lympoma, Diffuse Small Cleaved Cell, Diffuse Small Lymphocytic Lymphoma, Enteropathy-Type T-Cell Lymphoma, Extranodal Marginal Zone B-cell lymphoma, Extranodal NK/T-Cell Lymphoma—Nasal Type, Follicular Lymphoma, Follicular Small Cleaved Cell (Grade 1), Follicular Mixed Small Cleaved and Large Cell (Grade 2), Follicular Large Cell (Grade 3), Diffuse Small Cleaved Cell, Hepatosplenic T-Cell Lymphoma, Immunoblastic Lymphoma, Intermediate Differentiation Lymphoma, Intestinal T-Cell Lymphoma, Intravascular Large B-Cell Lymphoma, Intravascular Lymphomatosis, Large Cell Immunoblastic Lymphoma, Large Cell Lymphoma (LCL), Lymphoblastic Lymphoma, Lymphomatoid Granulomatosis, MALT Lymphoma, Mantle Cell Lymphoma (MCL), Mediastinal Large B-Cell Lymphoma, Monocytoid B-cell Lymphoma, Mycosis Fungoides, Cutaneous T-Cell Lymphoma, NK-Cell Lymphoma, Nodal Marginal Zone B-cell Lymphoma, Peripheral T-Cell Lymphoma (PTL), Pleomorphic T-Cell Lymphoma, Post-Transplantation Lymphoproliferative Disorder (PTLD), Precursor B-Lymphoblastic Lymphoma, Precursor T-Cell Lymphoblastic Lymphoma, Primary Central Nervous System Lymphoma (CNS), Primary Cutaneous Anaplastic Large Cell Lymphoma/Lymphomatoid Papulosis (CD30+), Primary Effusion Lymphoma, Primary Mediastinal B-cell Lymphoma, Sezary Syndrome, Small Lymphocytic Lymphoma, Small Non-Cleaved Cell Lymphoma (SNCL), Endemic Burkitt's Lymphoma, Sporadic Burkitt's Lymphoma, Non-Burkitt's Lymphoma, Splenic Marginal Zone Lymphoma, Subcutaneous Panniculitis-Like T-Cell Lymphoma, True Histiocytic Lymphoma, Waldenstrom's Macroglobulinemia, Lymphoplasmacytic Lymphoma, and the like.

Mantle cell lymphoma (MCL) is one type of non-Hodgkin's lymphoma that represents about 6% of all B-cell non-Hodgkin's lymphomas (B-NHL) (Jaffe, et al. ed., World health organization classification of tumours: Pathology and Genetics of Tumours of Haematopoietic and Lymphoid Tissues, Lyon: IARC Press, 2001). MCL typically involves a t(11;14)(q13;q32) translocation. Patients with MCL often have characteristics such as a blastic morphological variant, increased tumor cell proliferation, INK4a/ARF locus deletion and p53 mutation or protein overexpression (Campo et al., (1999) Semin Hematol 36:115-127). The disease has a median patient survival of three to four years. Several of the cell lines described herein, such as Rec-1, Jeko-1, Granta-519, and JVM-2 are mantle cell lymphomas.

5.3 Biomarkers

Provided herein are methods relating to the use of mRNAs or proteins as biomarkers to predict or ascertain the effectiveness of quinazolinone compound. mRNA or protein levels can be used to determine whether a potential quinazolinone compound is likely to be successful in cell models of disease.

A biological marker or “biomarker” is a substance whose detection indicates a particular biological state, such as, for example, the presence of cancer. In some embodiments, biomarkers can either be determined individually, or several biomarkers can be measured simultaneously.

In some embodiments, a “biomarker” indicates a change in the level of mRNA expression that may correlate with the risk or progression of a disease, or with the susceptibility of the disease to a given treatment. In some embodiments, the biomarker is a nucleic acid, such as a mRNA or cDNA.

In additional embodiments, a “biomarker” indicates a change in the level of polypeptide or protein expression that may correlate with the risk, susceptibility to treatment, or progression of a disease. In some embodiments, the biomarker can be a polypeptide or protein. Examples include, but are not limited to, SPARC, cyclin D1, p21, or a fragment thereof. The relative level of specific proteins can be determined by methods known in the art. For example, antibody based methods, such as an immunoblot, enzyme-linked immunosorbent assay (ELISA), or other methods can be used.

5.3.1 Use of SPARC mRNA or Protein as a Biomarker as an Early Indicator (or Predictor) of Treatment Success

Based, in part, on the finding that detectable increases in SPARC mRNA expression are visible in less than about 24 hours after administration of a quinazolinone compound in sensitive cell lines but not in resistant cell lines, SPARC mRNA or protein levels may be used as a biomarker for predicting the sensitivity of a cancer cell to a quinazolinone compound.

Thus, in some embodiments, the SPARC mRNA or protein biomarker can be used to predict the effectiveness of the treatment by a quinazolinone compound in a patient. In one embodiment, the level of the mRNA or protein is measured in a biological sample obtained from a potential patient. A quinazolinone compound is then administered directly to the patient. After a certain time, such as, for example, 24 hours, another sample is obtained, and the level of the SPARC mRNA or protein biomarker is compared to the level prior to administration of the compound, using, for example, RT-PCR based methods. An increased SPARC expression level after administration indicates the likelihood of effectiveness of the treatment in the patient.

Alternatively, SPARC can also be used as a biomarker for an in vitro assay to predict the success of the treatment with a quinazolinone compound, by taking a sample of cancer cells from the patient, culturing them in the presence or absence of a quinazolinone compound, and testing the cells for an increase in SPARC expression. Patients having cell samples that exhibit an increased expression of SPARC in the cell-based assay could then be treated with a quinazolinone compound.

5.3.2 Monitoring Progress of Patient Treatment Using SPARC mRNA or Protein Expression as a Biomarker

In addition to the initial prediction of the likelihood of treatment effectiveness in a patient with NHL, the progress of cancer treatment with a quinazolinone compound can be monitored using the expression of SPARC as a biomarker. Thus, in some embodiments, a method of assessing or monitoring the effectiveness of a treatment by a quinazolinone compound in a patient is provided. A sample is obtained from the patient, and the SPARC mRNA or protein level is measured to determine whether it is present at an increased or decreased level compared to the level prior to the initiation of treatment.

NHL patients can submit the cell sample by any desired means, such as, for example, a lymph node biopsy, bone marrow biopsy, or from a circulating tumor. Samples can be taken, for example, every day, once per week, twice per month, once a month, once every two months, quarterly, or yearly, as needed to follow the effectiveness of the treatment. In one embodiment, an increase in SPARC expression after administration indicates that the treatment protocol is effective. In another embodiment, a lack of an increase in SPARC expression after administration of the quinazolinone compound indicates that the treatment may not be effective in the particular patient, and that other treatment methods may need to be pursued. By following the SPARC mRNA or protein level, the treatment effectiveness can be monitored over time.

The mRNA or protein-based biomarkers can also be used to track and adjust individual patient treatment effectiveness. mRNA or protein-based biomarkers can be used to gather information needed to make adjustments in a patient's treatment, increasing or decreasing the dose of an agent as needed. For example, a patient receiving a quinazolinone compound can be tested using a SPARC mRNA or protein-based biomarker to see if the dosage is becoming effective, or if a more aggressive treatment plan may be needed.

5.3.3 Use of Cyclin D1 and p21 as Prediction Biomarkers

The finding of the differences in the patterns of cyclin D1 and p21 gene expression in the various cancer cell types allows for the prediction of likelihood of successful treatment with a quinazolinone compound by testing a biological sample from the patient, and comparing the baseline levels of cyclin D1 and p21 mRNA expression. Thus, the mRNAs can be used as biomarkers to predict the sensitivity to cancer treatment by administration of quinazolinone compounds. In particular, the mRNA levels of p21 and cyclin D1 can be used to determine whether a potential quinazolinone compound is likely to be successful in treating certain types of cancer, such as NHL (e.g., MCL). Thus, in one embodiment, a high level of cyclin D1 and a low level of p21 predicts the likelihood of increased sensitivity to quinazolinone compounds.

In some embodiments, this difference in gene expression is simply measured as the difference in PCR cycle time to reach a threshold fluorescence, or “dCT”. For example, if the CT of p21 minus the CT of cyclin D1 is greater than 0, there is a higher likelihood that the cell type will successfully respond to treatment with a quinazolinone compound. In some embodiments, the dCT is greater than 1, 2, 3, 4, 5, 6, 7, 8, 9, 10 or more.

In another embodiment, a dCT of p21 minus cyclin D1 that is less than 0 can predict that the quinazolinone compound will not be very effective in treating the patient.

In another embodiment, a method of predicting the sensitivity to treatment with an immunomodulatory compound in an NHL, specifically, a Mantle Cell Lymphoma (MCL), patient is provided. The method comprises obtaining a biological sample from the patient, optionally isolating or purifying mRNA from the biological sample, amplifying the mRNA transcripts by, e.g., RT-PCR, where a higher baseline level of Cyclin D1 (as assessed by, e.g., determining the cycle number at which the fluorescence passes the set threshold level (“CT”) of Cyclin D1 mRNA expression) indicates a higher likelihood that the cancer will be sensitive to treatment with an immunomodulatory compound.

Further, the expression of these genes can be used as biomarkers to monitor progress of treatment effectiveness in NHL patients that are receiving treatment with quinazolinone compounds with that of control samples.

5.3.4 Monitoring Patient Compliance Using p21, Cyclin D1, and SPARC mRNA or Protein Expression as a Biomarker

The p21, cyclin D1, and SPARC mRNA or protein biomarkers can additionally be used to track or perform quality control on human research trials or to monitor the patient compliance to a drug regimen by providing a means to confirm that the patient is receiving specific drug treatments. These biomarkers can be used in connection with, for example, the management of patient treatment, clinical trials, and cell-based research.

In one embodiment, the p21, cyclin D1, and SPARC mRNA or protein-based biomarkers can be used to track patient compliance during individual treatment regimes, or during clinical trials. Thus, in some embodiments, a method for assessing patient compliance with a drug treatment protocol is provided. A biological sample is obtained from the patient, and the levels of at least one of p21, cyclin D1, or SPARC mRNA or protein is measured and compared to that of a control untreated sample. An altered expression level of the mRNA or protein biomarker compared to that of an untreated control sample indicates compliance with the protocol.

For example, the expression of SPARC mRNA or protein can be followed at set intervals during a clinical trial to ensure that the patients included in the trial are taking the drugs as instructed. The treatment of individual patients can also be followed using the procedure. For example, when at least one of p21, cyclin D1, or SPARC mRNA or protein is measured, an altered level of the biomarker compared to that of an untreated control indicates at least partial patient compliance with the drug treatment protocol. An altered level of the mRNA or protein biomarker that is at a similar quantity to that of a positive control indicates the likelihood of full compliance with the treatment protocol.

5.4 Quinazolinone Compounds

The quinazolinone compounds are a group of compounds that can be useful to treat several types of human diseases, including certain cancers. As provided herein, these compounds can be effective in treating NHL. In some embodiments, a quinazolinone compound can be administered to a cell sample or to a patient, and the effectiveness of the treatment can be followed using mRNA or protein biomarkers as described herein. Quinazolinone compounds include those disclosed in U.S. patent application Ser. No. 11/904,551, and U.S. Provisional Application No. 60/995,676, both of which were filed Sep. 26, 2007 and are incorporated herein by reference in their entireties.

In one embodiment, provided herein are compounds of the formula (I):

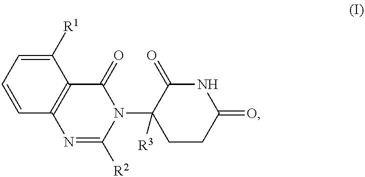

and pharmaceutically acceptable salts, solvates, and stereoisomers thereof, wherein:
R¹ is: hydrogen; halo; —(CH₂)ₙOH; (C₁-C₆)alkyl, optionally substituted with one or more halo; (C₁-C₆)alkoxy, optionally substituted with one or more halo; or —(CH₂)ₙNHR^{a}, wherein R^{a} is:
   hydrogen;
   (C₁-C₆)alkyl, optionally substituted with one or more halo;
   —(CH₂)ₙ-(6 to 10 membered aryl);
   —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
   —C(O)—(C₁-C₈)alkyl, wherein the alkyl is optionally substituted with one or more halo;
   —C(O)—(CH₂)ₙ—(C₃-C₁₀-cycloalkyl);
   —C(O)—(CH₂)ₙ—NR^{b}R^{c}, wherein R^{b} and R^{c} are each independently:
      hydrogen;
      (C₁-C₆)alkyl, optionally substituted with one or more halo;
      (C₁-C₆)alkoxy, optionally substituted with one or more halo; or
      6 to 10 membered aryl, optionally substituted with one or more of: halo; (C₁-C₆)alkyl, itself optionally substituted with one or more halo; or
         (C₁-C₆)alkoxy, itself optionally substituted with one or more halo;
   —C(O)—(CH₂)ₙ—O—(C₁-C₆)alkyl; or
   C(O)—(CH₂)ₙ—O—(CH₂)ₙ-(6 to 10 membered aryl);
R² is: hydrogen; —(CH₂)ₙOH; phenyl; —O—(C₁-C₆)alkyl; or (C₁-C₆)alkyl, optionally substituted with one or more halo;
R³ is: hydrogen; or (C₁-C₆)alkyl, optionally substituted with one or more halo;
   and
n is 0, 1, or 2.

In one embodiment, provided herein are compounds of the formula (II):

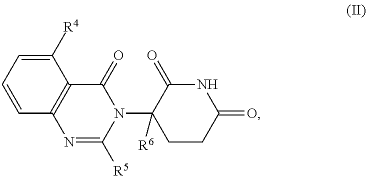

and pharmaceutically acceptable salts, solvates, and stereoisomers thereof, wherein:
R⁴ is: hydrogen; halo; —(CH₂)ₙOH; (C₁-C₆)alkyl, optionally substituted with one or more halo; or (C₁-C₆)alkoxy, optionally substituted with one or more halo;
R⁵ is: hydrogen; —(CH₂)ₙOH; phenyl; —O—(C₁-C₆)alkyl; or (C₁-C₆)alkyl, optionally substituted with one or more halo;
R⁶ is: hydrogen; or (C₁-C₆)alkyl, optionally substituted with one or more halo;
   and
n is 0, 1, or 2.

In one embodiment, R⁴ is hydrogen. In another embodiment, R⁴ is halo. In another embodiment, R⁴ is (C₁-C₆)alkyl, optionally substituted with one or more halo. In another embodiment, R⁴ is —(CH₂)ₙOH or hydroxyl. In another embodiment, R⁴ is (C₁-C₆)alkoxy, optionally substituted with one or more halo.

In one embodiment, R⁵ is hydrogen. In another embodiment, R⁵ is —(CH₂)ₙOH or hydroxyl. In another embodiment, R⁵ is phenyl. In another embodiment, R⁵ is —O—(C₁-C₆)alkyl, optionally substituted with one or more halo. In another embodiment, R⁵ is (C₁-C₆)alkyl, optionally substituted with one or more halo.

In one embodiment, R⁶ is hydrogen. In another embodiment, R⁶ is (C₁-C₆)alkyl, optionally substituted with one or more halo.

In one embodiment, n is 0. In another embodiment, n is 1. In another embodiment, n is 2.

Compounds provided herein encompass any of the combinations of R⁴, R⁵, R⁶ and n described above.

In one specific embodiment, R⁴ is methyl. In another embodiment, R⁴ is methoxy. In another embodiment, R⁴ is —CF₃. In another embodiment, R⁴ is F or Cl.

In another specific embodiment, R⁵ is methyl. In another embodiment, R⁵ is —CF₃.

Specific examples include, but are not limited to:

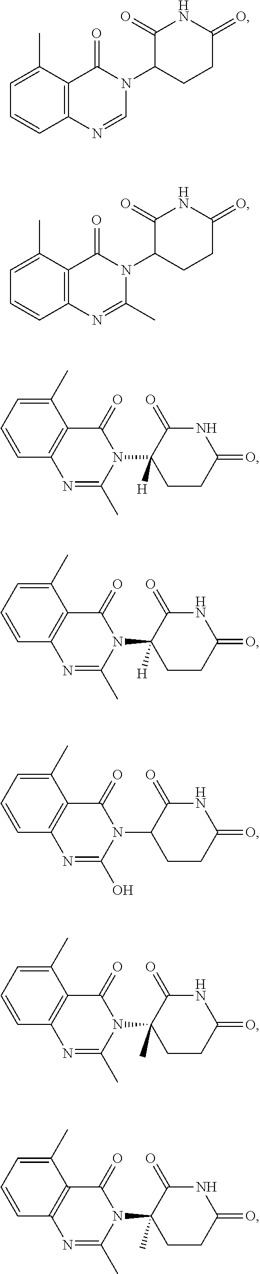

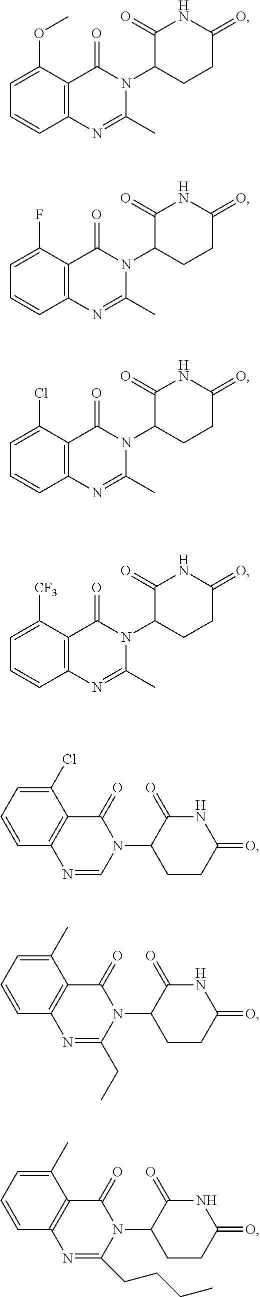

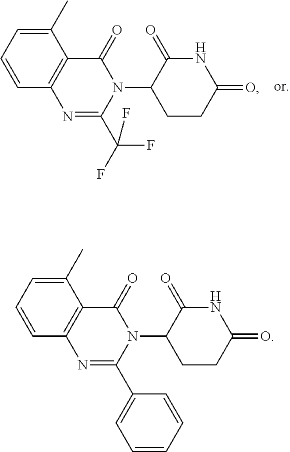

In another embodiment, provided herein are compounds of the formula (III):

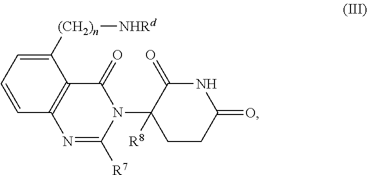

and pharmaceutically acceptable salts, solvates, and stereoisomers thereof, wherein:
R^{d} is:
   hydrogen;
   (C₁-C₆)alkyl, optionally substituted with one or more halo;
   —C(O)—(C₁-C₈)alkyl, wherein the alkyl is optionally substituted with one or more halo;
   —C(O)—(CH₂)ₙ—(C₃-C₁₀-cycloalkyl);
   —C(O)—(CH₂)ₙ—NR^{e}R^{f}, wherein R^{e} and Ware each independently:
      hydrogen;
      (C₁-C₆)alkyl, optionally substituted with one or more halo; or
      (C₁-C₆)alkoxy, optionally substituted with one or more halo; or
   —C(O)—(CH₂)ₙ—O—(C₁-C₆)alkyl.
R⁷ is: hydrogen; —(CH₂)ₙOH; phenyl; —O—(C₁-C₆)alkyl; or (C₁-C₆)alkyl, optionally substituted with one or more halo;
R⁸ is: hydrogen; or (C₁-C₆)alkyl, optionally substituted with one or more halo;
   and
n is 0, 1, or 2.

In one embodiment, R^{d} is hydrogen. In another embodiment, R^{d} is (C₁-C₆)alkyl, optionally substituted with one or more halo. In another embodiment, R^{d} is —C(O)—(C₁-C₈)alkyl. In another embodiment, R^{d} is —C(O)—(CH₂)ₙ—(C₃-C₁₀-cycloalkyl). In another embodiment, R^{d} is —C(O)—(CH₂)ₙ—NR^{e}R^{f}, wherein R^{e} and R^{f} are as described herein above. In another embodiment, R^{d} is —C(O)—(CH₂)ₙ—O—(CH₂)ₙ—(C₁-C₆)alkyl.

In one embodiment, R⁷ is hydrogen. In another embodiment, R⁷ is —(CH₂)ₙOH or hydroxyl. In another embodiment, R⁷ is phenyl. In another embodiment, R⁷ is —O—(C₁-C₆)alkyl, optionally substituted with one or more halo. In another embodiment, R⁷ is (C₁-C₆)alkyl, optionally substituted with one or more halo.

In one embodiment, R⁸ is hydrogen. In another embodiment, R⁸ is (C₁-C₆)alkyl, optionally substituted with one or more halo.

In one embodiment, n is 0. In another embodiment, n is 1. In another embodiment, n is 2.

Compounds provided herein encompass any of the combinations of R^{d}, R⁷, R⁸ and n described above.

In one specific embodiment, R⁷ is methyl. In another embodiment, R^{d} is —C(O)—(C₁-C₆)alkyl. In another embodiment, R^{d} is NH₂. In another embodiment, R^{d} is —C(O)—CH₂—O—(C₁-C₆)alkyl.

Specific examples include, but are not limited to:

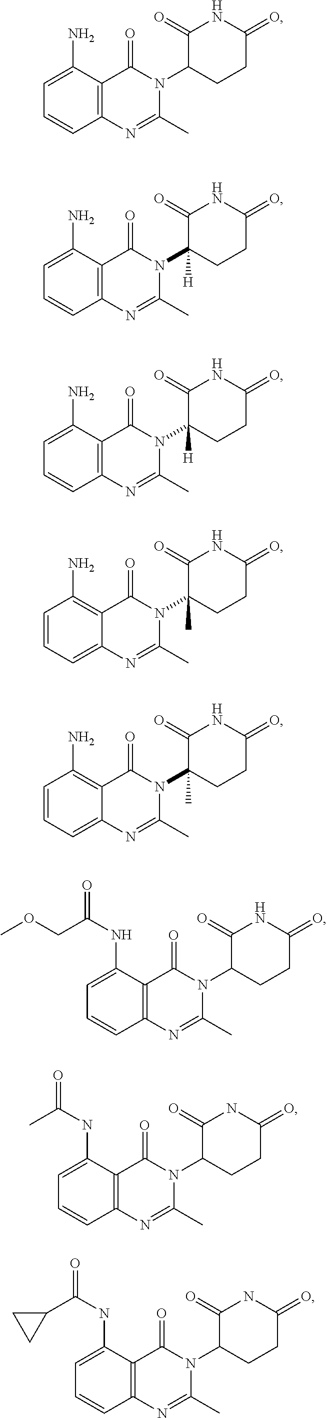

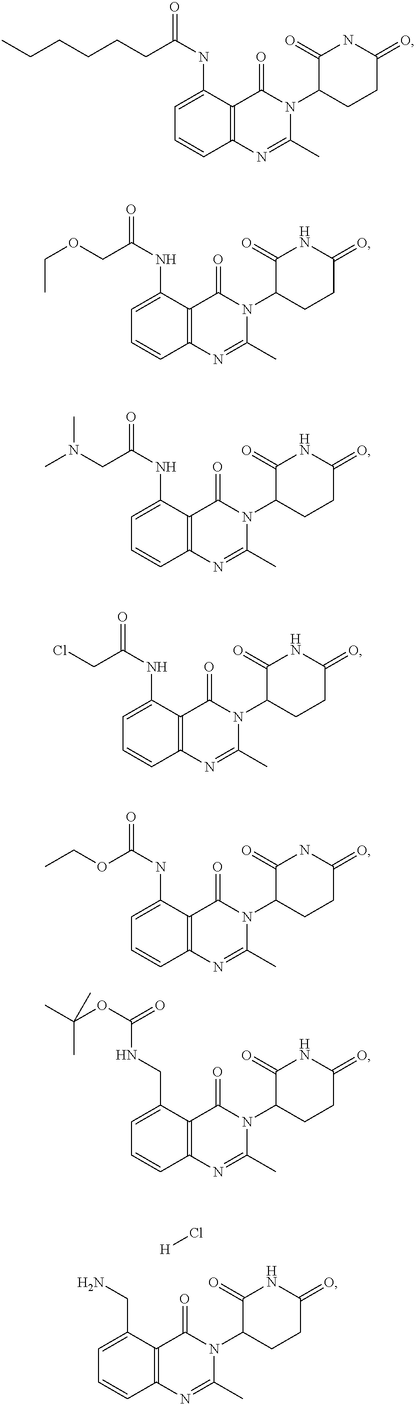

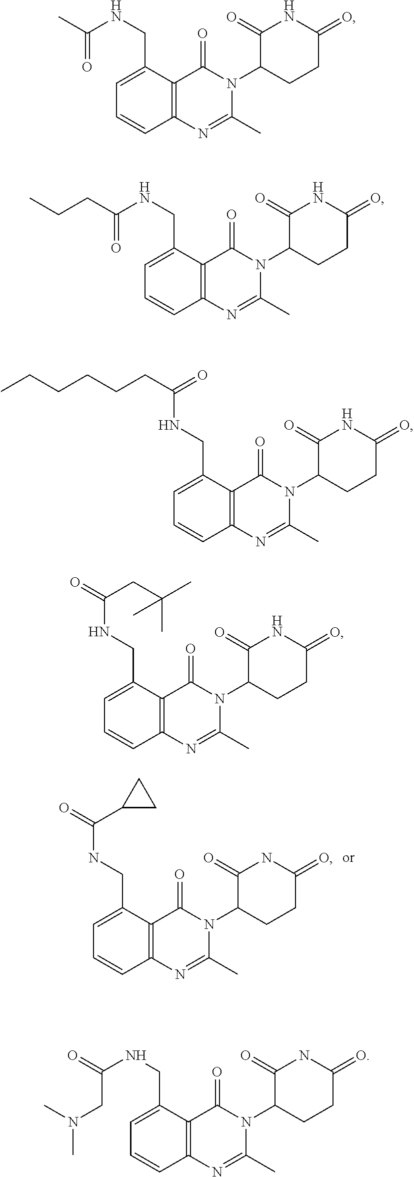

In another embodiment, In another embodiment, provided herein are compounds of the formula (IV):

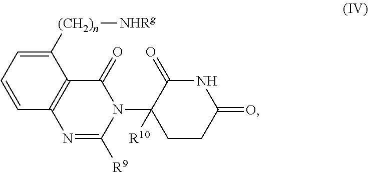

and pharmaceutically acceptable salts, solvates, and stereoisomers thereof, wherein:
R^{g} is:
   —(CH₂)ₙ-(6 to 10 membered aryl);
   —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
   —C(O)—(CH₂)ₙ—NHR^{h}, wherein R^{h} is:
      6 to 10 membered aryl, optionally substituted with one or more of: halo; (C₁-C₆)alkyl, itself optionally substituted with one or more halo; or (C₁-C₆)alkoxy, itself optionally substituted with one or more halo; or
   —C(O)—(CH₂)ₙ—O—(CH₂)ₙ-(6 to 10 membered aryl);
R⁹ is: hydrogen; —(CH₂)ₙOH; phenyl; —O—(C₁-C₆)alkyl; or (C₁-C₆)alkyl, optionally substituted with one or more halo;
R¹⁰ is: hydrogen; or (C₁-C₆)alkyl, optionally substituted with one or more halo;
   and
n is 0, 1, or 2.

In one embodiment, R^{g} is —(CH₂)ₙ-(6 to 10 membered aryl). In another embodiment, R^{g} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{g} is —C(O)—(CH₂)ₙ—NHR^{h}, wherein R^{h} is 6 to 10 membered aryl, optionally substituted as described above. In another embodiment, R^{g} is —C(O)—(CH₂)ₙ—O—(CH₂)ₙ-(6 to 10 membered aryl).

In one embodiment, R⁹ is hydrogen. In another embodiment, R⁹ is —(CH₂)ₙOH or hydroxyl. In another embodiment, R⁹ is phenyl. In another embodiment, R⁹ is —O—(C₁-C₆)alkyl, optionally substituted with one or more halo. In another embodiment, R⁹ is (C₁-C₆)alkyl, optionally substituted with one or more halo.

In one embodiment, R¹⁰ is hydrogen. In another embodiment, R¹⁰ is (C₁-C₆)alkyl, optionally substituted with one or more halo.

In one embodiment, n is 0. In another embodiment, n is 1. In another embodiment, n is 2.

Compounds provided herein encompass any of the combinations of R^{g}, R⁹, R¹⁰ and n described above.

In one specific embodiment, R⁹ is methyl. In another embodiment, R^{g} is —C(O)-phenyl or —C(O)—CH₂-phenyl, wherein the phenyl is optionally substituted with methyl, —CF₃, and/or halo. In another embodiment, R^{g} is —C(O)—NH-phenyl, wherein the phenyl is optionally substituted with methyl, —CF₃, and/or halo.

Specific compounds include, but are not limited to:

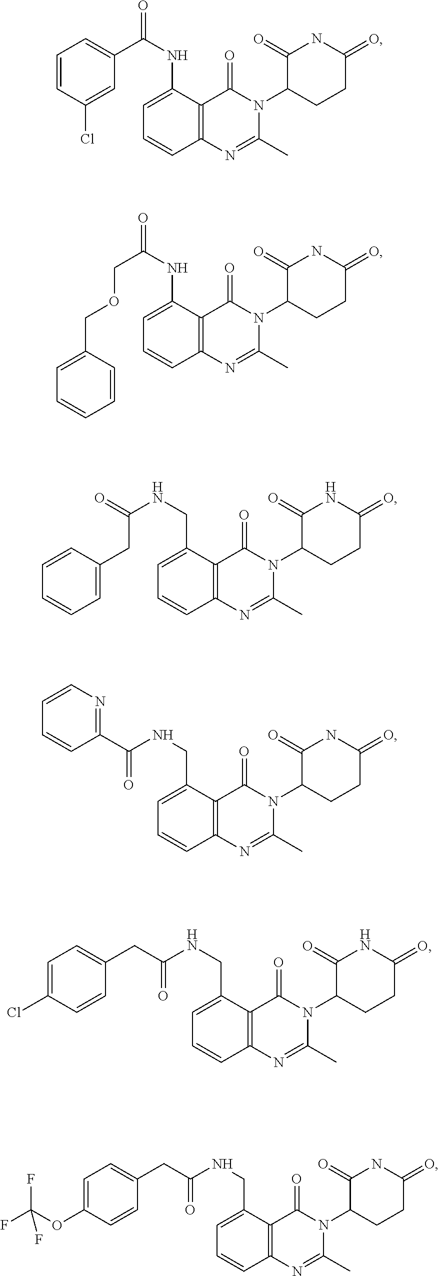

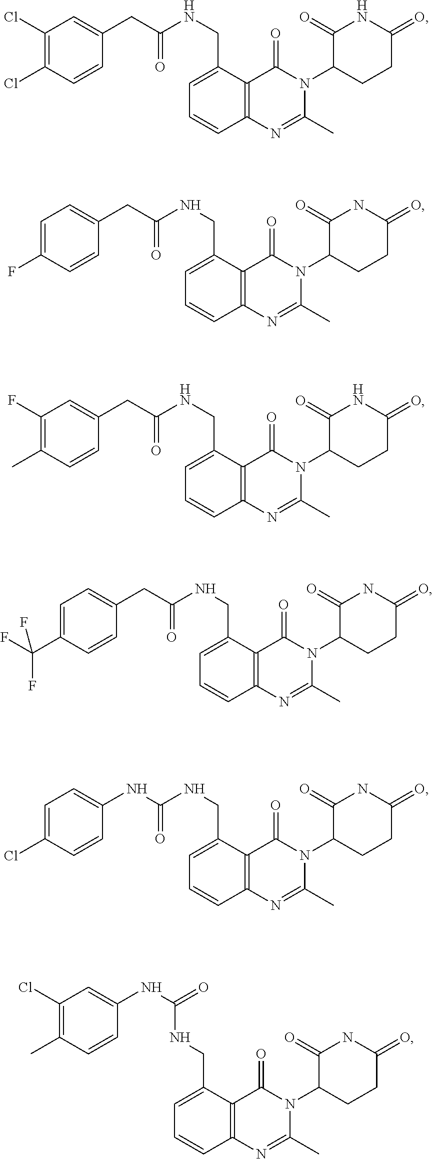

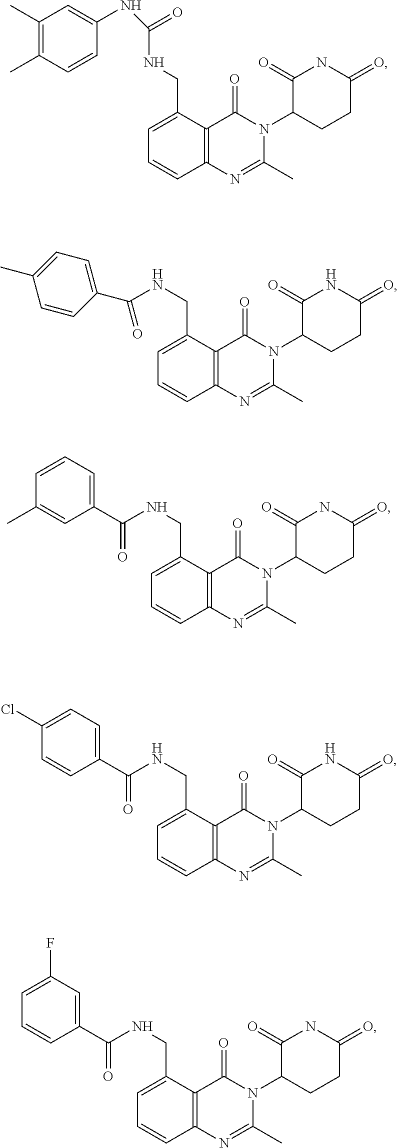

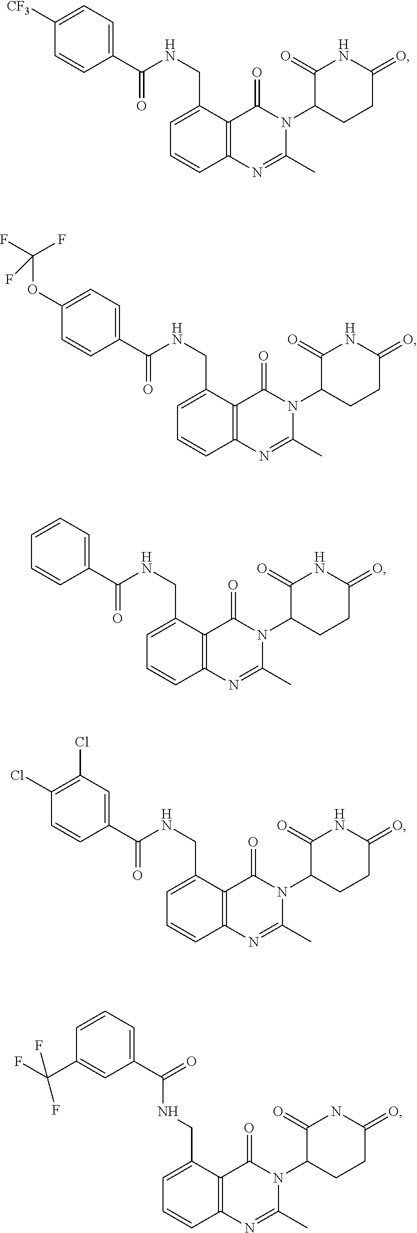

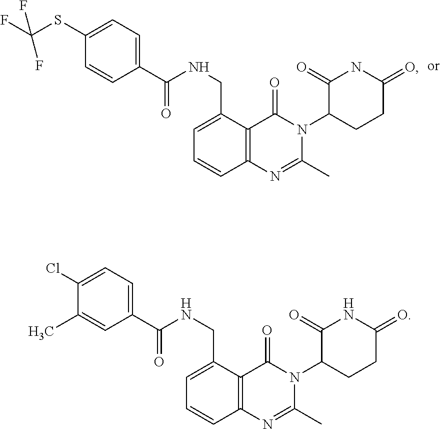

In another embodiment, the compounds provided herein for use in the pharmaceutical compositions and methods have the formula (A):

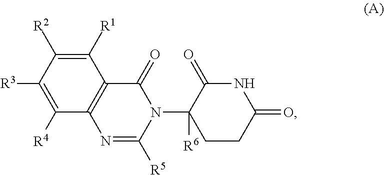

and pharmaceutically acceptable salts, solvates, and stereoisomers thereof, wherein:
R¹ is hydrogen;
each of R², R³, and R⁴ is independently: hydrogen; halo; —(CH₂)ₙOH; (C₁-C₆)alkyl, optionally substituted with one or more halo; (C₁-C₆)alkoxy, optionally substituted with one or more halo; or
   —(CH₂)ₙNHR^{a}, wherein R^{a} is:
      hydrogen;
      (C₁-C₆)alkyl, optionally substituted with one or more halo;
      —(CH₂)ₙ-(6 to 10 membered aryl);
      —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, said alkyl itself optionally substituted with one or more halo; or
         (C₁-C₆)alkoxy; said alkoxy itself optionally substituted with one or more halo;
      —C(O)—(C₁-C₈)alkyl, wherein the alkyl is optionally substituted with one or more halo;
      —C(O)—(CH₂)ₙ—(C₃-C₁₀-cycloalkyl);
      —C(O)—(CH₂)ₙ—NR^{b}R^{c}, wherein R^{b} and R^{c} are each independently:
         hydrogen;
         (C₁-C₆)alkyl, optionally substituted with one or more halo;
         (C₁-C₆)alkoxy, optionally substituted with one or more halo; or
         6 to 10 membered aryl, optionally substituted with one or more of: halo; (C₁-C₆)alkyl, itself optionally substituted with one or more halo; or (C₁-C₆)alkoxy, itself optionally substituted with one or more halo;
      —C(O)—(CH₂)ₙ—O—(C₁-C₆)alkyl; or
      —C(O)—(CH₂)ₙO—(CH₂)ₙ-(6 to 10 membered aryl); or
two of R¹-R⁴ together can form a 5 or 6 membered ring, optionally substituted with one or more of: halo; (C₁-C₆)alkyl, optionally substituted with one or more halo; and (C₁-C₆)alkoxy, optionally substituted with one or more halo;
R⁵ is: hydrogen; —(CH₂)ₙOH; phenyl; —O—(C₁-C₆)alkyl; or (C₁-C₆)alkyl, optionally substituted with one or more halo;
R⁶ is: hydrogen; or (C₁-C₆)alkyl, optionally substituted with one or more halo; and
n is 0, 1, or 2.

In another embodiment, provided herein are compounds of formula (B):

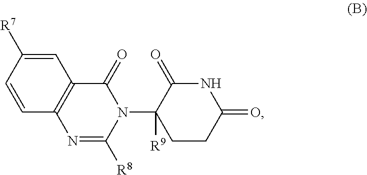

and pharmaceutically acceptable salts, solvates, and stereoisomers thereof, wherein:
R⁷ is: hydrogen; halo; —(CH₂)ₙOH; (C₁-C₆)alkyl, optionally substituted with one or more halo; (C₁-C₆)alkoxy, optionally substituted with one or more halo; or
   —(CH₂)ₙNHR^{d}, wherein R^{d} is:
      hydrogen;
      (C₁-C₆)alkyl, optionally substituted with one or more halo;
      —(CH₂)ₙ-(6 to 10 membered aryl);
      —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
      —C(O)—(C₁-C₈)alkyl, wherein the alkyl is optionally substituted with one or more halo;
      —C(O)—(CH₂)ₙ—(C₃-C₁₀-cycloalkyl);
      —C(O)—(CH₂)ₙ—NR^{e}R^{f}, wherein R^{e} and R^{f} are each independently:
         hydrogen;
         (C₁-C₆)alkyl, optionally substituted with one or more halo;
         (C₁-C₆)alkoxy, optionally substituted with one or more halo; or
         6 to 10 membered aryl, optionally substituted with one or more of: halo; (C₁-C₆)alkyl, itself optionally substituted with one or more halo; or (C₁-C₆)alkoxy, itself optionally substituted with one or more halo;
      —C(O)—(CH₂)ₙ—O—(C₁-C₆)alkyl; or
      —C(O)—(CH₂)ₙ—O—(CH₂)ₙ-(6 to 10 membered aryl);
R⁸ is: hydrogen; —(CH₂)ₙOH; phenyl; —O—(C₁-C₆)alkyl; or (C₁-C₆)alkyl, optionally substituted with one or more halo;
R⁹ is: hydrogen; or (C₁-C₆)alkyl, optionally substituted with one or more halo; and
n is 0, 1, or 2.

In another embodiment, provided herein are compounds of formula (C):

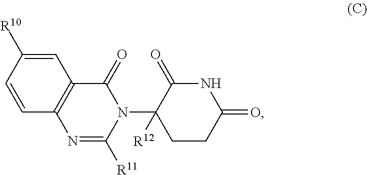

and pharmaceutically acceptable salts, solvates, and stereoisomers thereof, wherein:
R¹⁰ is: hydrogen; halo; —(CH₂)ₙOH; (C₁-C₆)alkyl, optionally substituted with one or more halo; or (C₁-C₆)alkoxy, optionally substituted with one or more halo;
R¹¹ is: hydrogen; —(CH₂)ₙOH; phenyl; —O—(C₁-C₆)alkyl; or (C₁-C₆)alkyl, optionally substituted with one or more halo;
R¹² is: hydrogen; or (C₁-C₆)alkyl, optionally substituted with one or more halo; and
n is 0, 1, or 2.

In one embodiment, R¹⁰ is hydrogen. In another embodiment, R¹⁰ is halo. In another embodiment, R¹⁰ is (C₁-C₆)alkyl, optionally substituted with one or more halo. In another embodiment, R¹⁰ is —(CH₂)ₙOH or hydroxyl. In another embodiment, R¹⁰ is (C₁-C₆)alkoxy, optionally substituted with one or more halo.

In one embodiment, R¹¹ is hydrogen. In another emdodiment, R¹¹ is —(CH₂)ₙOH or hydroxyl. In another emdodiment, R¹¹ is phenyl. In another emdodiment, R¹¹ is —O—(C₁-C₆)alkyl, optionally substituted with one or more halo. In another emdodiment, R¹¹ is (C₁-C₆)alkyl, optionally substituted with one or more halo.

In one embodiment, R¹² is hydrogen. In another embodiment, R¹² is (C₁-C₆)alkyl, optionally substituted with one or more halo.

In one embodiment, n is 0. In another embodiment, n is 1. In another embodiment, n is 2.

Compounds provided herein encompass any of the combinations of R¹⁰, R¹¹, R¹² and n described above.

In one specific embodiment, R¹⁰ is halo. In another embodiment, R¹⁰ is hydroxyl. In another embodiment, R¹⁰ is methyl.

In another specific embodiment, R¹¹ is hydrogen. In another embodiment, R¹¹ is methyl.

In another specific embodiment, R¹² is hydrogen. In another embodiment, R¹² is methyl.

Specific compounds include, but are not limited to:

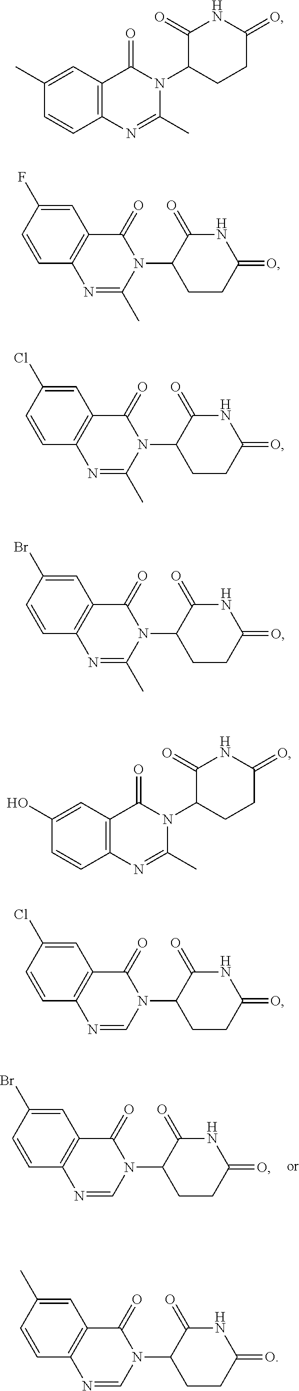

In another embodiment, provided herein are compounds of formula (D):

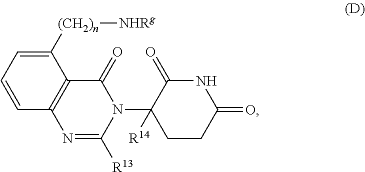

and pharmaceutically acceptable salts, solvates, and stereoisomers thereof, wherein:
R^{g} is:
   hydrogen;
   (C₁-C₆)alkyl, optionally substituted with one or more halo;
   —(CH₂)ₙ-(6 to 10 membered aryl);
   —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
   —C(O)—(C₁-C₈)alkyl, wherein the alkyl is optionally substituted with one or more halo;
   —C(O)—(CH₂)ₙ—(C₃-C₁₀-cycloalkyl);
   —C(O)—(CH₂)ₙ—NR^{h}Rⁱ, wherein R^{h} and Rⁱ are each independently:
      hydrogen;
      (C₁-C₆)alkyl, optionally substituted with one or more halo;
      (C₁-C₆)alkoxy, optionally substituted with one or more halo; or
      6 to 10 membered aryl, optionally substituted with one or more of: halo; (C₁-C₆)alkyl, itself optionally substituted with one or more halo; or (C₁-C₆)alkoxy, itself optionally substituted with one or more halo;
   —C(O)—(CH₂)ₙ—O—(C₁-C₆)alkyl; or
   —C(O)—(CH₂)ₙ—O—(CH₂)ₙ-(6 to 10 membered aryl);
R¹³ is: hydrogen; —(CH₂)ₙOH; phenyl; —O—(C₁-C₆)alkyl; or (C₁-C₆)alkyl, optionally substituted with one or more halo;
R¹⁴ is: hydrogen; or (C₁-C₆)alkyl, optionally substituted with one or more halo; and
n is 0, 1, or 2.

In one embodiment, R^{g} is hydrogen. In another embodiment, R^{g} is (C₁-C₆)alkyl, optionally substituted with one or more halo. In another embodiment, R^{g} is —(CH₂)ₙ-(6 to 10 membered aryl). In another embodiment, R^{g} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{g} is —C(O)—(C₁-C₈)alkyl, wherein the alkyl is optionally substituted with one or more halo. In another embodiment, R^{g} is —C(O)—(CH₂)ₙ—(C₃-C₁₀-cycloalkyl). In another embodiment, R^{g} is —C(O)—(CH₂)ₙ—NR^{h}Rⁱ, wherein R^{h} and Rⁱ are as described above. In another embodiment, R^{g} is —C(O)—(CH₂)ₙ—O—(C₁-C₆)alkyl. In another embodiment, R^{g} is —C(O)—(CH₂)ₙ—O—(CH₂)ₙ-(6 to 10 membered aryl).

In one embodiment, R¹³ is hydrogen. In another embodiment, R¹³ is —(CH₂)ₙOH or hydroxyl. In another embodiment, R¹³ is phenyl. In another embodiment, R¹³ is —O—(C₁-C₆)alkyl, optionally substituted with one or more halo. In another embodiment, R¹³ is (C₁-C₆)alkyl, optionally substituted with one or more halo.

In one embodiment, R¹⁴ is hydrogen. In another embodiment, R¹⁴ is (C₁-C₆)alkyl, optionally substituted with one or more halo.

In one embodiment, n is 0. In another embodiment, n is 1. In another embodiment, n is 2.

Compounds provided herein encompass any of the combinations of R^{g}, R¹³, R¹⁴ and n described above.

In one specific embodiment, R^{g} is hydrogen, and n is 0 or 1. In another embodiment, R^{g} is —C(O)—(C₁-C₆)alkyl. In another embodiment, R^{g} is —C(O)-phenyl, optionally substituted with one or more methyl, halo, and/or (C₁-C₆)alkoxy.

In another specific embodiment, R¹³ is methyl. In another embodiment, R¹⁴ is hydrogen.

Specific compounds include, but are not limited to:

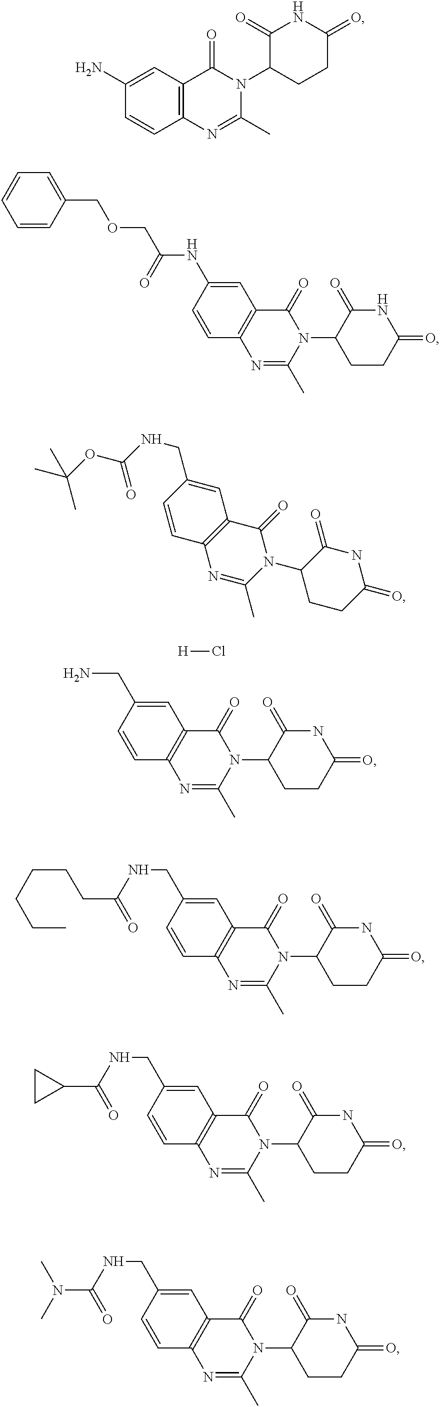

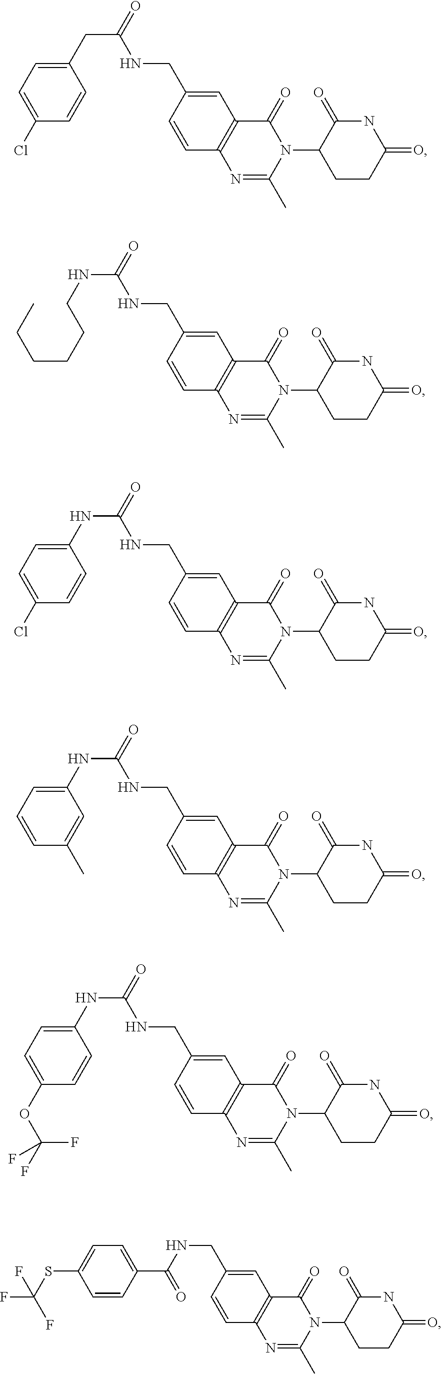

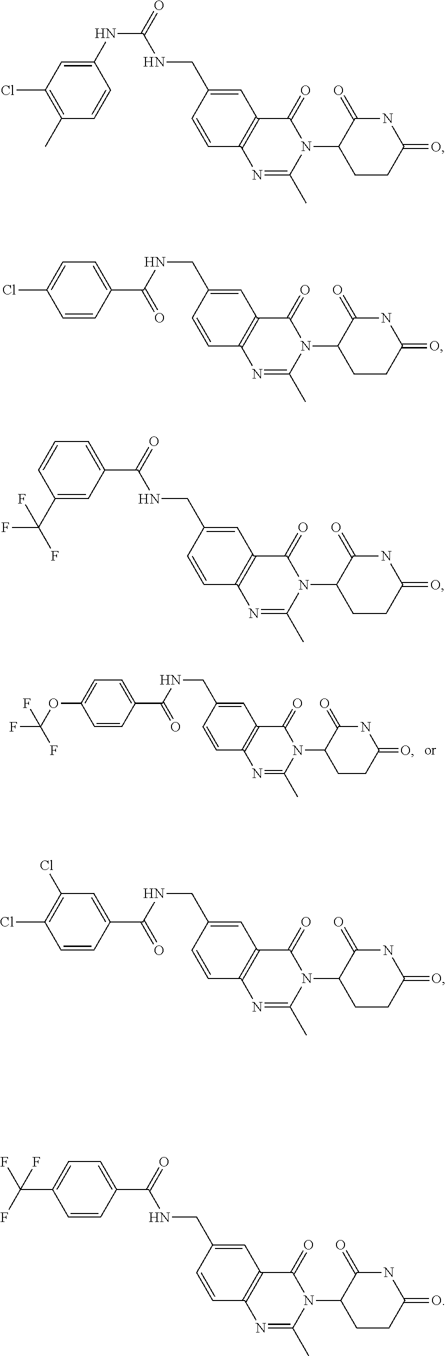

In another embodiment, provided herein are compounds of formula (E):

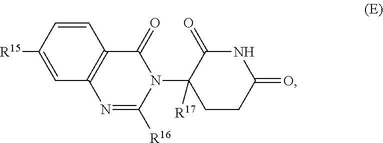

and pharmaceutically acceptable salts, solvates, and stereoisomers thereof, wherein:
R¹⁵ is: hydrogen; halo; —(CH₂)ₙOH; (C₁-C₆)alkyl, optionally substituted with one or more halo; (C₁-C₆)alkoxy, optionally substituted with one or more halo; or
   —(CH₂)ₙNHR^{j}, wherein R^{j} is:
      hydrogen;
      (C₁-C₆)alkyl, optionally substituted with one or more halo;
      —(CH₂)ₙ-(6 to 10 membered aryl);
      —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
      —C(O)—(C₁-C₈)alkyl, wherein the alkyl is optionally substituted with one or more halo;
      —C(O)—(CH₂)ₙ—(C₃-C₁₀-cycloalkyl);
      —C(O)—(CH₂)ₙ—NR^{k}R¹, wherein R^{k} and R¹ are each independently:
         hydrogen;
         (C₁-C₆)alkyl, optionally substituted with one or more halo;
         (C₁-C₆)alkoxy, optionally substituted with one or more halo; or
         6 to 10 membered aryl, optionally substituted with one or more of: halo; (C₁-C₆)alkyl, itself optionally substituted with one or more halo; or (C₁-C₆)alkoxy, itself optionally substituted with one or more halo;
      —C(O)—(CH₂)ₙ—O—(C₁-C₆)alkyl; or
      —C(O)—(CH₂)ₙ—O—(CH₂)ₙ-(6 to 10 membered aryl);
R¹⁶ is: hydrogen; —(CH₂)ₙOH; phenyl; —O—(C₁-C₆)alkyl; or (C₁-C₆)alkyl, optionally substituted with one or more halo;
R¹⁷ is: hydrogen; or (C₁-C₆)alkyl, optionally substituted with one or more halo; and
n is 0, 1, or 2.

In one embodiment, R¹⁵ is hydrogen. In another embodiment, R¹⁵ is halo. In another embodiment, R¹⁵ is (C₁-C₆)alkyl, optionally substituted with one or more halo. In another embodiment, R¹⁵ is —(CH₂)ₙOH or hydroxyl. In another embodiment, R¹⁵ is (C₁-C₆)alkoxy, optionally substituted with one or more halo.

In one embodiment, R¹⁵ is —(CH₂)ₙNHR^{j}. In one embodiment, wherein R¹⁵ is —(CH₂)ₙNHR^{j}, R^{j} is hydrogen. In another embodiment, R^{j} is (C₁-C₆)alkyl, optionally substituted with one or more halo. In another embodiment, R^{j} is —(CH₂)ₙ-(6 to 10 membered aryl). In another embodiment, R^{j} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{j} is —C(O)—(C₁-C₈)alkyl, wherein the alkyl is optionally substituted with one or more halo. In another embodiment, R^{j} is —C(O)—(CH₂)ₙ—(C₃-C₁₀-cycloalkyl). In another embodiment, R^{j} is —C(O)—(CH₂)ₙ—NR^{k}R^{l}, wherein R^{k} and R^{l} are as described above. In another embodiment, R^{j} is —C(O)—(CH₂)ₙ—O—(C₁-C₆)alkyl. In another embodiment, R^{j} is —C(O)—(CH₂)ₙ—O—(CH₂)ₙ-(6 to 10 membered aryl).

In one embodiment, R¹⁶ is hydrogen. In another embodiment, R¹⁶ is —(CH₂)ₙOH or hydroxyl. In another embodiment, R¹⁶ is phenyl. In another embodiment, R¹⁶ is —O—(C₁-C₆)alkyl, optionally substituted with one or more halo. In another embodiment, R¹⁶ is (C₁-C₆)alkyl, optionally substituted with one or more halo.

In one embodiment, R¹⁷ is hydrogen. In another embodiment, R¹⁷ is (C₁-C₆)alkyl, optionally substituted with one or more halo.

In one embodiment, n is 0. In another embodiment, n is 1. In another embodiment, n is 2.

Compounds provided herein encompass any of the combinations of R¹⁵, R¹⁶, R¹⁷ and n described above.

In one specific embodiment, R¹⁵ is methyl. In another embodiment, R¹⁵ is halo. In another embodiment, R¹⁵ is —CF₃. In another embodiment, R¹⁵ is —(CH₂)ₙNHR^{j}.

In one specific embodiment wherein R¹⁵ is —(CH₂)ₙNHR^{j}, R^{j} is hydrogen, and n is 0 or 1. In another embodiment wherein R¹⁵ is —(CH₂)ₙNHR^{j}, R^{j} is —C(O)—(O)—(C₁-C₆)alkyl.

In one specific embodiment, R¹⁶ is hydrogen. In another embodiment, R¹⁶ is methyl. In another specific embodiment, R¹⁷ is hydrogen or methyl.

Specific compounds include, but are not limited to:

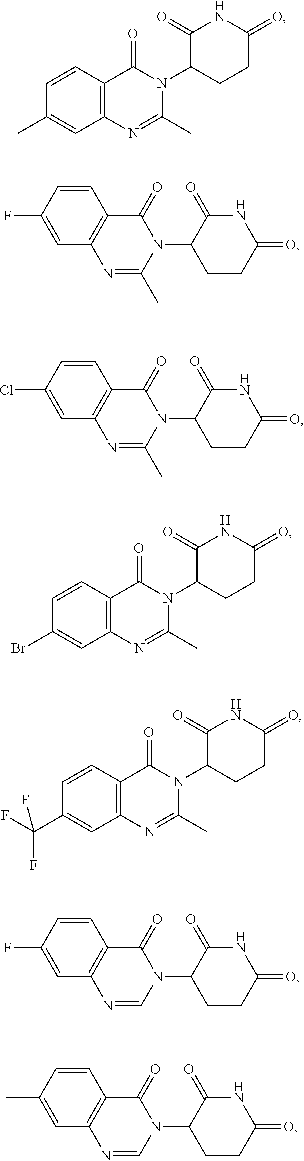

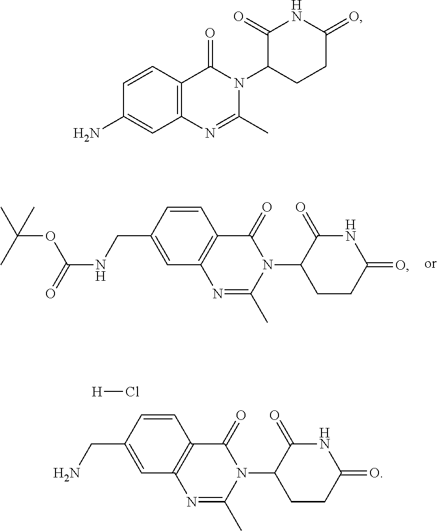

In another embodiment, provided herein are compounds of formula (F):

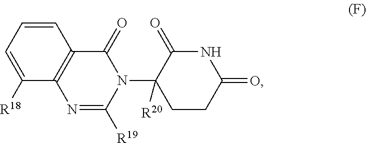

and pharmaceutically acceptable salts, solvates, and stereoisomers thereof, wherein:
R¹⁸ is: hydrogen; halo; —(CH₂)ₙOH; (C₁-C₆)alkyl, optionally substituted with one or more halo; (C₁-C₆)alkoxy, optionally substituted with one or more halo; or —(CH₂)ₙNHR^{m}, wherein R^{m} is:
   hydrogen;
   (C₁-C₆)alkyl, optionally substituted with one or more halo;
   —(CH₂)ₙ-(6 to 10 membered aryl);
   —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
   —C(O)—(C₁-C₈)alkyl, wherein the alkyl is optionally substituted with one or more halo;
   —C(O)—(CH₂)ₙ—(C₃-C₁₀-cycloalkyl);
   —C(O)—(CH₂)ₙ—NRⁿR^{o}, wherein Rⁿ and R^{o} are each independently:
      hydrogen;
      (C₁-C₆)alkyl, optionally substituted with one or more halo;
      (C₁-C₆)alkoxy, optionally substituted with one or more halo; or
      6 to 10 membered aryl, optionally substituted with one or more of: halo; (C₁-C₆)alkyl, itself optionally substituted with one or more halo; or (C₁-C₆)alkoxy, itself optionally substituted with one or more halo;
   —C(O)—(CH₂)ₙ—O—(C₁-C₆)alkyl; or
   —C(O)—(CH₂)ₙ—O—(CH₂)ₙ-(6 to 10 membered aryl);
R¹⁹ is: hydrogen; —(CH₂)ₙOH; phenyl; —O—(C₁-C₆)alkyl; or (C₁-C₆)alkyl, optionally substituted with one or more halo;
R²⁰ is: hydrogen; or (C₁-C₆)alkyl, optionally substituted with one or more halo; and
n is 0, 1, or 2.

In one embodiment, R¹⁸ is hydrogen. In another embodiment, R¹⁸ is halo. In another embodiment, R¹⁸ is (C₁-C₆)alkyl, optionally substituted with one or more halo. In another embodiment, R¹⁸ is —(CH₂)ₙOH or hydroxyl. In another embodiment, R¹⁸ is (C₁-C₆)alkoxy, optionally substituted with one or more halo.

In one embodiment, R¹⁸ is —(CH₂)ₙNHR^{m}. In one embodiment, wherein R²⁸ is —(CH₂)ₙNHR^{s}, R^{s} is hydrogen. In another embodiment, R^{m} is (C₁-C₆)alkyl, optionally substituted with one or more halo. In another embodiment, R^{M} is —(CH₂)ₙ-(6 to 10 membered aryl). In another embodiment, R^{m} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{s} is —C(O)—(C₁-C₈)alkyl, wherein the alkyl is optionally substituted with one or more halo. In another embodiment, R^{m} is —C(O)—(CH₂)ₙ—(C₃-C₁₀-cycloalkyl). In another embodiment, R^{m} is —C(O)—(CH₂)ₙ—NRⁿR^{o}, wherein Rⁿ and R^{o} are as described above. In another embodiment, R^{m} is —C(O)—(CH₂)ₙ—O—(C₁-C₆)alkyl. In another embodiment, R^{m} is —C(O)—(CH₂)ₙ—O—(CH₂)ₙ-(6 to 10 membered aryl).

In one embodiment, R¹⁹ is hydrogen. In another embodiment, R¹⁹ is —(CH₂)ₙOH or hydroxyl. In another embodiment, R¹⁹ is phenyl. In another embodiment, R¹⁹ is —O—(C₁-C₆)alkyl, optionally substituted with one or more halo. In another embodiment, R¹⁹ is (C₁-C₆)alkyl, optionally substituted with one or more halo.

In one embodiment, R²⁰ is hydrogen. In another embodiment, R²⁰ is (C₁-C₆)alkyl, optionally substituted with one or more halo.

In one embodiment, n is 0. In another embodiment, n is 1. In another embodiment, n is 2.

Compounds provided herein encompass any of the combinations of R¹⁸, R¹⁹, R²⁰ and n described above.

In one specific embodiment, R¹⁸ is methyl. In another embodiment, R¹⁸ is halo. In another embodiment, R¹⁸ is hydroxyl. In another embodiment, R¹⁸ is —CF₃.

In one specific embodiment, R¹⁹ is hydrogen. In another embodiment, R¹⁹ is methyl. In another specific embodiment, R²⁰ is hydrogen.

Specific compounds include, but are not limited to:

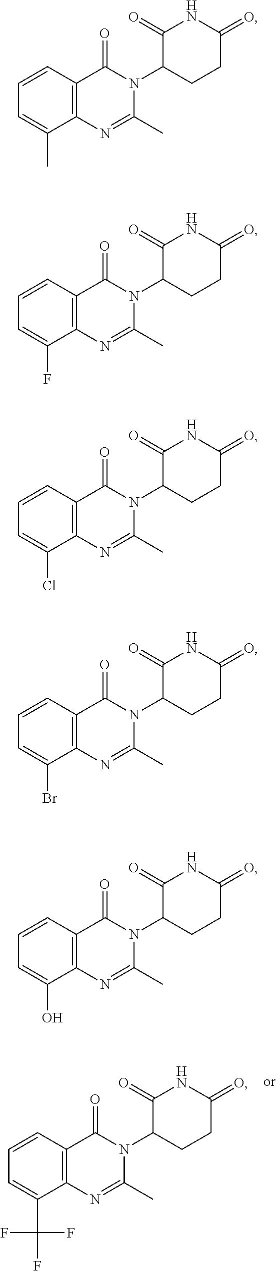

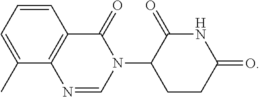

In another embodiment, provided herein are compounds of formula (G):

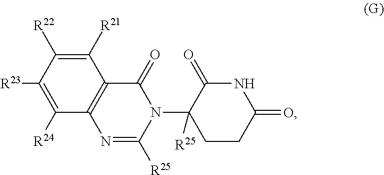

and pharmaceutically acceptable salts, solvates, and stereoisomers thereof, wherein:
R²¹ is hydrogen;
R²², R²³, and R²⁴ are each independently: halo; —(CH₂)ₙOH; (C₁-C₆)alkyl, optionally substituted with one or more halo; (C₁-C₆)alkoxy, optionally substituted with one or more halo; or
two of R²¹-R²⁴ together form a 5 to 6 membered ring, optionally substituted with one or more of: halo; (C₁-C₆)alkyl, optionally substituted with one or more halo; and (C₁-C₆)alkoxy, optionally substituted with one or more halo;
R²⁵ is: hydrogen; —(CH₂)ₙOH; phenyl; —O—(C₁-C₆)alkyl; or (C₁-C₆)alkyl, optionally substituted with one or more halo;
R²⁶ is: hydrogen; or (C₁-C₆)alkyl, optionally substituted with one or more halo; and
n is 0, 1, or 2.

In one embodiment, two of R²²-R²⁴ are halo. In another embodiment, two of R²²-R²⁴ are (C₁-C₆)alkyl, optionally substituted with one or more halo. In another embodiment, two of R²²-R²⁴ are (C₁-C₆)alkoxy, optionally substituted with one or more halo.

In another embodiment, one of R²²-R²⁴ are is halo, and another one of R²²-R²⁴ is (C₁-C₆)alkyl, optionally substituted with one or more halo. In another embodiment, one of R²²-R²⁴ is halo, and another one of R²²-R²⁴ is (C₁-C₆)alkoxy, optionally substituted with one or more halo. In another embodiment, one of R²²-R²⁴ is (C₁-C₆)alkoxy, optionally substituted with one or more halo, and another one of R²²-R²⁴ is (C₁-C₆)alkyl, optionally substituted with one or more halo.

In another embodiment, two of R²²-R²⁴ together form a 5 to 6 membered ring. In one specific embodiment, R²² and R²³ together form a 5 to 6 membered ring. In one specific embodiment, R²² and R²³ together form phenyl ring. In another embodiment, the ring formed by R²² and R²³ is optionally substituted with one or more of: halo; (C₁-C₆)alkyl, optionally substituted with one or more halo; and (C₁-C₆)alkoxy, optionally substituted with one or more halo.

In one embodiment, R²⁵ is hydrogen. In another embodiment, R²⁵ is —(CH₂)ₙOH or hydroxyl. In another embodiment, R²⁵ is phenyl. In another embodiment, R²⁵ is —O—(C₁-C₆)alkyl, optionally substituted with one or more halo. In another embodiment, R²⁵ is (C₁-C₆)alkyl, optionally substituted with one or more halo.

In one embodiment, R²⁶ is hydrogen. In another embodiment, R²⁶ is (C₁-C₆)alkyl, optionally substituted with one or more halo.

In one embodiment, n is 0. In another embodiment, n is 1. In another embodiment, n is 2.

Compounds provided herein encompass any of the combinations of R²¹, R²², R²³, R²⁴, R²⁵, R²⁶, and n described above.

Specific compounds include, but are not limited to:

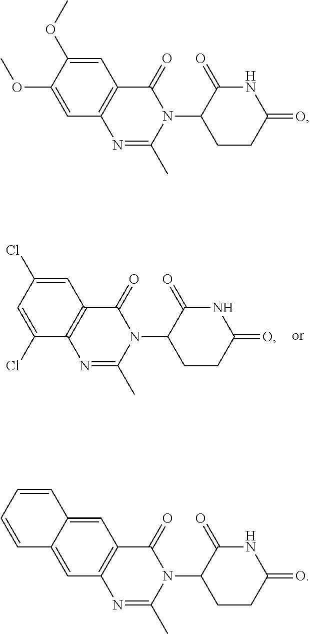

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. In one embodiment, suitable are hydrochloric, hydrobromic, phosphoric, and sulfuric acids.

As used herein, and unless otherwise specified, the term “solvate” means a compound that further includes a stoichiometric or non-stoichiometric amount of solvent bound by non-covalent intermolecular forces. Where the solvent is water, the solvate is a hydrate.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein, and unless otherwise specified, the terms “biohydrolyzable carbamate,” “biohydrolyzable carbonate,” “biohydrolyzable ureide” and “biohydrolyzable phosphate ” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, carbamates that include lower alkylamine, substituted ethylenediamine, aminoacid, hydroxyalkylamine, heterocyclic and heteroaromatic amine, and polyether amine moieties.

As used herein, and unless otherwise specified, the term “stereoisomer” encompasses all enantiomerically/stereomerically pure and enantiomerically/stereomerically enriched compounds provided herein.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

As used herein, and unless otherwise indicated, the term “alkyl” refers to a saturated straight chain or branched hydrocarbon having a number of carbon atoms as specified herein. Representative saturated straight chain alkyls include -methyl, -ethyl, -n-propyl, -n-butyl, -n-pentyl, and -n-hexyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The term “alkyl” also encompasses cycloalkyl.

As used herein, and unless otherwise specified, the term “cycloalkyl” means a specie of alkyl containing from 3 to 15 carbon atoms, without alternating or resonating double bonds between carbon atoms. It may contain from 1 to 4 rings. Examples of unsubstituted cycloalkyls include, but are not limited to, cyclopropyl, cyclobutyl, cyclopentyl, cyclohexyl, and adamantyl. A cycloalkyl may be substituted with one or more of the substituents.

As used herein, the term “aryl” means a carbocyclic aromatic ring containing from 5 to 14 ring atoms. The ring atoms of a carbocyclic aryl group are all carbon atoms. Aryl ring structures include compounds having one or more ring structures such as mono-, bi-, or tricyclic compounds as well as benzo-fused carbocyclic moieties such as 5,6,7,8-tetrahydronaphthyl and the like. Specifically, the aryl group is a monocyclic ring or bicyclic ring. Representative aryl groups include phenyl, anthracenyl, fluorenyl, indenyl, azulenyl, phenanthrenyl and naphthyl.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

5.5 Methods of Detecting mRNA or Protein Levels in a Sample

Any suitable method of detecting differences the levels of mRNA or protein biomarkers, such as SPARC, cyclin D1, p21, can be used. In some embodiments, the biomarker to be detected is an mRNA molecule. In other embodiments, the method of measuring gene or protein expression can involve methods such as cDNA hybridization, flow cytometry, immunofluorescence, immunoblots, ELISAs or microspotted-antibody immunofluorescence assays, an antibody-based dipstick assay, cytometric bead arrays, or other common mRNA or protein detecting methods.

5.5.1 Methods of Detecting mRNA Levels in a Sample

Several methods of detecting or quantitating mRNA levels are known in the art. Exemplary methods include, but are not limited to, northern blots, ribonuclease protection assays, PCR-based methods, and the like. When the biomarker is an mRNA molecule, the mRNA sequence, e.g., SPARC, cyclin D1, p21 mRNA, or a fragment thereof, can be used to prepare a probe that is at least partially complementary. The probe can then be used to detect the mRNA sequence in a sample, using any suitable assay, such as PCR-based methods, Northern blotting, a dipstick assay, and the like.

In other embodiments, a nucleic acid assay for testing for the activity of a quinazolinone compound in a biological sample can be prepared. An assay typically contains a solid support and at least one nucleic acid contacting the support, where the nucleic acid corresponds to at least a portion of an mRNA that has altered expression during a treatment by a quinazolinone compound in a patient, such as SPARC, cyclin D1, or p21 mRNA. The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′,5′ dichloro 2′,7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

In some embodiments, the mRNA sequences comprise at least one mRNA selected from the group consisting of SPARC mRNA, cyclin D1 mRNA, p21 mRNA, or a fragment thereof. The nucleic acids may be present in specific, addressable locations on a solid support; each corresponding to at least a portion of mRNA sequences that are differentially expressed upon treatment of a quinazolinone compound in a cell or a patient.

A typical mRNA assay method can contain the steps of: 1) obtaining surface-bound subject probes; 2) hybridization of a population of mRNAs to the surface-bound probes under conditions sufficient to provide for specific binding; 3) post-hybridization washes to remove nucleic acids not bound in the hybridization; and 4) detection of the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g. under conditions sufficient to provide for specific binding of target mRNAs in the sample to the probes) are described in Kallioniemi et al., Science 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

5.5.2 PCR-Based Methods of Detecting mRNA Biomarkers

Other methods, such as PCR-based methods, can also be used to follow the expression of the SPARC, cyclin D1, or p21 biomarkers. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, Real-Time Reverse Transcription-PCR (qRT-PCR) can be used for both the detection and quantification of RNA targets (Bustin, et al., 2005, Clin. Sci., 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using a 7500 Real-Time PCR System Sequence Detection software v1.3 using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

In some embodiments, the ratio of cyclin D1 to p21 expression can be analyzed using the above-described real-time PCR methods. One method of measuring this ratio is by use of the comparative CT relative quantification calculation method, which is known to those of skill in the art. In this method, quantitation of the amount of cDNA in the original sample is generally measured where the amplification of cDNAs becomes exponential with respect to the PCR cycle number. This is generally at the beginning of the upturn of the curve. Thus, in some embodiments, the measurement occurs at the cycle number at which the increase in fluorescence (and therefore cDNA) is exponential. This is shown by a horizontal threshold line on the cycle number vs. fluorescence curve and the point at which the fluorescence crosses the threshold is called the CT.

For example, the relative expression of p21 and cyclin D1 may be measured. Further, in some embodiments, the difference between CTs of cyclin D1 and p21 (dCT) may be used as an indicator of efficacy. In one embodiment, cancer cell types that are likely to be responsive to a quinazolinone compound can readily be predicted by having a dCT of greater than 0. In one embodiment, these cancer cell types are mantle cell lymphoma cells.

5.5.3 Methods of Detecting Polypeptide or Protein Biomarkers

When the biomarker is a protein, such as SPARC, Cyclin D1, or p21 protein, several protein detection and quantitation methods can be used to measure the presence of the biomarker. Any suitable protein quantitation method can be used. In some embodiments, antibody-based methods are used. Exemplary methods that can be used include but are not limited to immunoblotting (western blot), enzyme-linked immunosorbent assay (ELISA), immunohistochemistry, flow cytometry, cytometric bead array, mass spectroscopy, and the like. Several types of ELISA are commonly used, including direct ELISA, indirect ELISA, and sandwich ELISA.

5.6 Biological Samples

Any suitable sample can be used to assess the mRNA or protein biomarkers provided herein. In some embodiments, the biological sample is whole blood, partially purified blood, a PBMC, a tissue biopsy, an RNA or protein extract, a cell extract, a cell lysate, a cell, a cell culture, a cell line, a tissue, an oral tissue, a gastrointestinal tissue, an organ, an organelle, a biological fluid, a blood sample, a urine sample, a skin sample, a plurality of samples from a clinical trial, or the like. In an embodiment, the sample is a lymph node biopsy, a bone marrow biopsy, or a sample of peripheral blood tumor cells. The sample can be a crude sample, or can be purified to various degrees prior to storage, processing, or measurement.

Samples for mRNA or protein assessment can be taken during any desired intervals. For example, samples can be taken hourly, twice per day, daily, weekly, monthly, every other month, yearly, or the like. The sample can be tested immediately, or can be stored for later testing.

The samples can be purified prior to testing. In some embodiments, the mRNA or protein can be isolated from the remaining cell contents prior to testing. Control samples can be taken from various sources. In some embodiments, control samples are taken from the patient prior to treatment. A cell-based assay can utilize a control cell culture, for example, that has not been treated with the test compound.

5.7 Screening for Effective Quinazolinone Compounds Using mRNA or Protein Biomarkers

In some embodiments, a method of screening for effective quinazolinone compounds for treating several types of NHL can be obtained using the methods provided herein. For example, an NHL cell type is chosen and cultured. Baseline SPARC mRNA or protein is measured. The cell (or cells) are then contacted with a drug candidate, or a plurality of drug candidates. After an incubation period to allow for gene expression to occur, the level of SPARC or its mRNA is measured and compared to that of a similar untreated cell. The mRNA or protein levels are analyzed to determine whether the treated sample exhibits increased SPARC expression. Drug candidates that exhibit a pattern of increased SPARC expression can then be chosen for further studies to elucidate the activity of the candidate compound.

5.8 Kits for Detecting mRNA Biomarkers

In some embodiments, a kit for detecting the SPARC, cyclin D1, and p21 mRNA biomarkers can be prepared. The kits can include, for example, a probe or probe set comprising oligonucleotides that can bind to the mRNA biomarker(s) of interest for a given disease, compound, or other parameter. Washing solutions, reagents for performing a hybridization assay, mRNA isolation or purification means, detection means, as well as positive and negative controls can also be included. The kit can also include instructions for using the components of the kit. The kit can be tailored for in-home use, clinical use, or research use.

5.9 Kits for Detecting Polypeptide or Protein Biomarkers

In some embodiments, a kit for detecting the SPARC, cyclin D1, and p21 protein levels can be prepared. The kits can include, for example, a dipstick coated with an antibody that recognizes the protein, washing solutions, reagents for performing the assay, protein isolation or purification means, detection means, as well as positive and negative controls. The kit can also include instructions for using the components of the kit. The kit can be tailored for in-home use, clinical use, or research use.

### 6. EXAMPLES

The examples below are carried out using standard techniques, which are well known and routine to those of skill in the art, except where otherwise described in detail. The examples are illustrative, but do not limit the invention.

As detailed below, the effect of the administration of a quinazolinone compound on several types of NHL cells was determined after 3 days using 3H-thymidine incorporation, microbead array technology and real time PCR.

6.1 Antiproliferative Properties of Quinazolinone Compounds in Certain NHL Cell Lines

To better understand how the effectiveness of quinazolinone compound administration varies among different cancer cell types, the quinazolinone compound 3-(2,5-dimethyl-4-oxo-4H-quinazolin-3-yl)-piperidine-2,6-dione was tested in vitro for its anti-proliferative effect in several NHL cell lines after 3 days of treatment.

NHL cell proliferation was assessed by 3H-thymidine incorporation assay. Briefly, cells were cultured in 96 well cell culture plates in complete RPMI-1640 medium in the presence and absence of drugs. Following incubation at 37° C. for 3 days, 1μCi 3H-thymidine was added to each well for the last 5 hours of incubation. The 3H incorporation of each well was then measured. The six tested cell lines were Namalwa, Rec-1, Jeko-1, Granta-519, JVM-2, and DB.

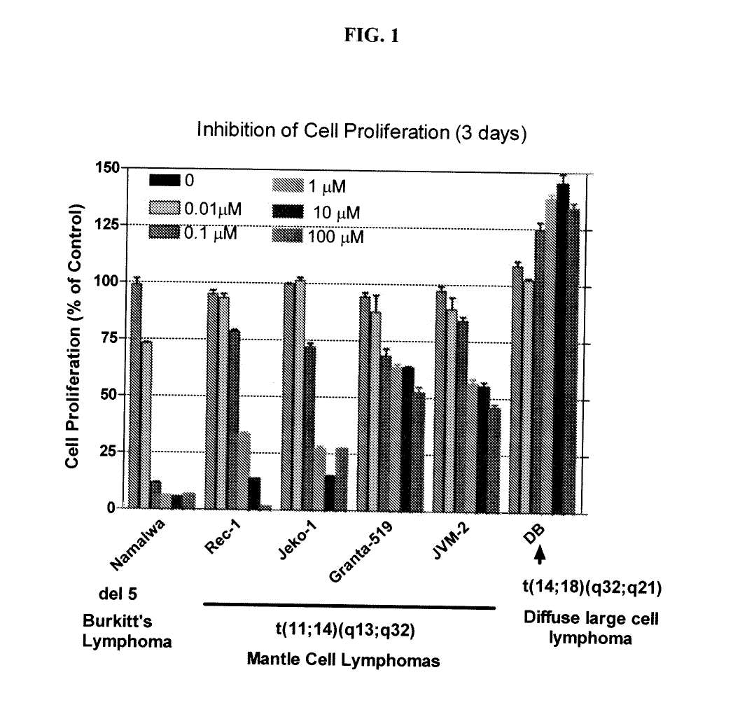

As shown in FIG. 1, quinazolinone compound 3-(2,5-dimethyl-4-oxo-4H-quinazolin-3-yl)-piperidine-2,6-dione demonstrated anti-proliferative activity against several types of NHL cells.

Upon cytogenetic analysis, it was found that Namalwa cells have a 5q deletion. Rec-1, Jeko-1, Granta-519 and JVM-2 cells have a t(11;14)(q13;q32), which is the hallmark for mantle cell lymphoma (MCL) cell lines; and DB cells have the t(14;18)(q32;q21), which is characteristic of follicular lymphoma. The anti-proliferative was shown to be dose dependent in the ranges tested (0, 0.01, 0.1, 1, 10, and 100 μM), especially in MCL cell lines.

6.2 Effect of the Administration of Quinazolinone Compound on mRNA Level of SPARC in Certain NHL Cells

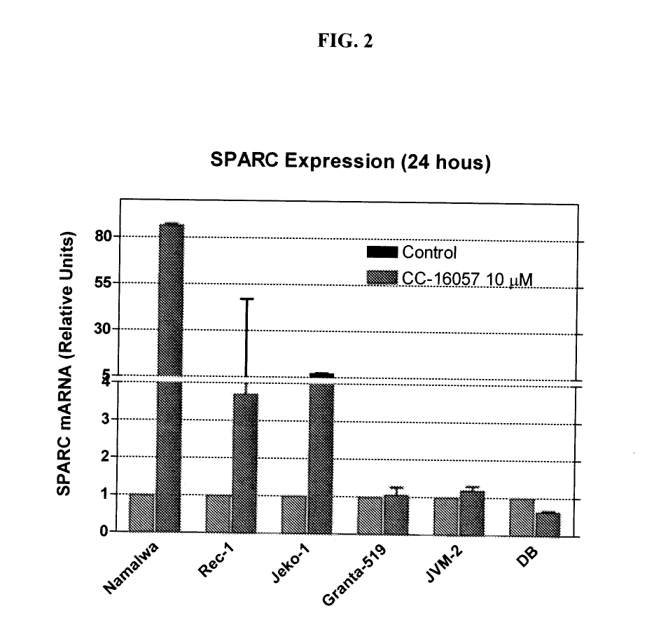

The effect of the administration of 3-(2,5-dimethyl-4-oxo-4H-quinazolin-3-yl)-piperidine-2,6-dione in various NHL cells was measured after 24 hours of incubation, using real-time RT-PCR (FIG. 2). The results show that 3-(2,5-dimethyl-4-oxo-4H-quinazolin-3-yl)-piperidine-2,6-dione increased the expression of SPARC mRNA in sensitive NHL cells.

The order of sensitivity of the various NHL cell lines to The order of sensitivity of the various NHL cell lines to 1-oxo-2-(2,6-dioxopiperidin-3-yl)-4-aminoisoindoline is Namalwa (Burkitt's lymphoma)>Jeko-1>Rec-1>JVM-2>Granta-519 (all Mantle Cell Lymphomas, MCL)>DB (Diffuse Large B Cell Lymphoma, DLBCL. All four of the treated MCL lines contained the characteristic t(11;14) chromosomal translocation that results in overexpression of the cell cycle protein cyclin D1. The DB cell line contained the t(14;18) chromosomal translocation that results in overexpression of the anti-apoptotic protein bc12. This DB cell line actually responded to 3-(2,5-dimethyl-4-oxo-4H-quinazolin-3-yl)-piperidine-2,6-dione with an increased rate of cell proliferation. Thus t(14;18) may be a negative prognostic factor in predicting clinical response to 3-(2,5-dimethyl-4-oxo-4H-quinazolin-3-yl)-piperidine-2,6-dione. Thus, SPARC gene expression can be used as a biomarker of NHL or MCL tumor response to quinazolinone compounds such as 3-(2,5-dimethyl-4-oxo-4H-quinazolin-3-yl)-piperidine-2,6-dione.

6.3 Gene Expression Markers that Predict Sensitivity of Mantle Cell Lymphoma to Treatment with Quinazolinone Compounds

The finding that expression levels of certain cancer related genes differ between various cancer cell lines led to a more detailed investigation to determine whether certain cancer cell types are more sensitive to specific quinazolinone compounds than others. Thus, patterns of constitutive (baseline) gene expression that predict sensitivity of NHL tumors to 1-oxo-2-(2,6-dioxopiperidin-3-yl)-4-aminoisoindoline therapy were sought. Baseline expression of specific genes of various NHL cell lines is measured by quantitative real-time polymerase chain reaction (qRT-PCR) using total RNA.

The following exemplary procedures can be used. Upon purification of total RNA, real-time PCR is performed with 100 ng of total RNA, using an Applied Biosystems 7500 instrument (Applied Biosystems, Foster City, Calif.) using TaqMan Sequence Detection chemistry which uses a fluorogenic probe (FAM) to enable the detection of a specific PCR product as it accumulates during PCR. Samples are prepared in triplicate in 50 μl reaction volumes. The 50 μl reactions consist of 25 μl 2× TaqMan PCR master mix, 2.5 μl of 20× gene expression assay, 10 μl of RNA (500 ng) and 12.5 μl water. Glyceraldehyde-3-phosphate dehydrogenase (GAPDH) is used as the endogenous control to ensure equal RNA amounts in each sample.

TaqMan® Gene Expression Assays are pre-formulated gene expression assays for rapid, reliable detection and quantification of human, mouse and rat mRNA transcripts. The PCR program used may be: 50° C. for 2 minutes, 95° C. for 10 minutes, 40 cycles of 95° C. for 15 seconds, 60° C. for 1 minute. Data is analyzed using 7500 Real-Time PCR System Sequence Detection software v1.3 using the comparative CT relative quantification calculation method. The output is expressed as a fold-change of expression levels. The threshold level is selected to be automatically determined by the software and is set to be above the baseline but sufficiently low to be within the exponential growth region of an amplification curve. The cycle number at which the fluorescence signal associated with a particular amplicon accumulation crosses the threshold is referred to as the CT.

The cell cycle protein cyclin D1, particularly in combination with the cyclin dependent kinase CdK4, stimulates progression through the cell cycle, resulting in an increase in cell proliferation. The p21 protein inhibits CdK proteins, typically resulting in the inhibition of cell cycle progression. P21 may also inhibit DNA replication in S phase cells.

The differences in gene expression in cyclin D1:p21 ratios upon treatment by a quinazolinone compound can be used as markers to predict whether a patient with a given type of NHL will be likely to be effectively treated with a quinazolinone compound. To predict the likelihood of a successful treatment outcome with a specific quinazolinone compound, baseline cyclin D1 and p21 gene expression can be monitored by qRT-PCR in lymph node or bone marrow biopsy, or in peripheral blood tumor cells, from patients with NHL and in particular MCL, as a means of predicting which patient will be most likely to benefit from a quinazolinone compound therapy.

As an example, a patient with NHL (e.g., MCL) is identified and a lymph node biopsy is taken. Baseline levels of cyclin D1 and p21 gene expression are measured. The probability of a successful treatment with a quinazolinone compound is determined by comparing the levels of cyclin D1 and p21 expression. A patient with a high baseline cyclin D1 and low baseline p21 gene expression is given a high probability of successful treatment with a quinazolinone compound, and is assigned to a treatment protocol that involves daily oral administration of a quinazolinone compound. Alternatively, a patient with a low baseline cyclin D1 and high baseline p21 gene expression is assigned to a lower probability of successful treatment with quinazolinone compound, and is thus assigned to a different treatment therapy.

From the foregoing, it will be appreciated that, although specific embodiments of the invention have been described herein for the purpose of illustration, various modifications may be made without deviating from the spirit and scope of the invention.

1. A method of predicting tumor response to treatment in a Non-Hodgkin's Lymphoma (NHL) patient, comprising:
obtaining tumor cells from the patient;
culturing the cells in the presence or absence of a quinazolinone compound;
measuring SPARC expression in the tumor cells; and
comparing the levels of SPARC expression level in tumor cells cultured in the presence of a quinazolinone compound to those in tumor cells cultured in the absence of the compound;
wherein an increased level of SPARC expression in the presence of a quinazolinone compound indicates the likelihood of an effective patient tumor response to quinazolinone compound.

2. A method of monitoring tumor response to treatment in a Non-Hodgkin's Lymphoma (NHL) patient, comprising:
obtaining a biological sample from the patient;
measuring SPARC expression in the biological sample;
administering a quinazolinone compound to the patient;
thereafter obtaining a second biological sample from the patient;
measuring SPARC expression in the second biological sample; and
comparing the levels of SPARC expression;
wherein an increased level of SPARC expression after treatment indicates the likelihood of an effective tumor response.

3. A method for monitoring patient compliance with a drug treatment protocol, comprising:
obtaining a biological sample from said patient;
measuring the expression level of at least one of p21 and SPARC in said sample; and
determining if the expression level is increased in the patient sample compared to the expression level in a control untreated sample;
wherein an increased expression indicates patient compliance with said drug treatment protocol.

4. The method of claim 1, wherein the expression is mRNA expression or protein expression.

5. The method of claim 1, wherein the expression in the treated sample increases by about 1.5×, 2.0×, 3×, 5×, or more.

6. The method of claim 1, wherein the quinazolinone compound is 3-(2,5-dimethyl-4-oxo-4H-quinazolin-3-yl)-piperidine-2,6-dione.

7. A method of predicting the sensitivity to treatment with a quinazolinone compound in a Mantle Cell Lymphoma (MCL) patient, comprising:
obtaining a biological sample from the patient;
optionally isolating or purifying mRNA from the biological sample; and
comparing by PCR the cycle number at which the fluorescence passes the set threshold level (CT) of P21 and Cyclin D1 mRNA expression;
wherein a greater difference between P21 CT and Cyclin D1 CT (dCT) indicates a higher likelihood that the MCL will be sensitive to treatment with a quinazolinone compound.

8. The method of claim 7, wherein the difference between P21 CT and Cyclin D1 CT is greater than 0.

9. The method of claim 8, wherein the difference between P21 CT and Cyclin D1 CT is greater than about 1, 2, 3, 4, 5, 6, 7, 8, 9, or 10.

10. The method of claim 7, wherein the quinazolinone compound is 3-(2,5-dimethyl-4-oxo-4H-quinazolin-3-yl)-piperidine-2,6-dione.

11-17. (canceled)

18. The method of claim 2, wherein the expression is mRNA expression or protein expression.

19. The method of claim 2, wherein the expression in the treated sample increases by about 1.5×, 2.0×, 3×, 5×, or more.

20. The method of claim 2, wherein the quinazolinone compound is 3-(2,5-dimethyl-4-oxo-4H-quinazolin-3-yl)-piperidine-2,6-dione.

21. The method of claim 3, wherein the expression is mRNA expression or protein expression.

22. The method of claim 3, wherein the expression in the treated sample increases by about 1.5×, 2.0×, 3×, 5×, or more.

23. The method of claim 3, wherein the quinazolinone compound is 3-(2,5-dimethyl-4-oxo-4H-quinazolin-3-yl)-piperidine-2,6-dione.
